(19) United States
(12) Patent Application Publication
(10) Pub. No.: US 2025/0391517 A1
(43) Pub. Date: Dec. 25, 2025

(54) AN OPTICAL DENSITY MEASUREMENT AND TESTING DEVICE

(71) Applicants: Russell Gary Latimer, Brentwood, TN (US); Willard Watson Young, III, Brentwood, TN (US); David John Vigerust, Brentwood, TN (US)

(72) Inventors: Russell Gary Latimer, Brentwood, TN (US); Willard Watson Young, III, Brentwood, TN (US); David John Vigerust, Brentwood, TN (US)

(21) Appl. No.: 19/308,266

(22) Filed: Aug. 24, 2025

Related U.S. Application Data
(63) Continuation-in-part of application No. 18/227,982, filed on Jul. 31, 2023.
Application No. 18/227,982 is a continuation of application No. 17/692,493, filed on Mar. 11, 2022, now abandoned.

(51) Int. Cl. G16C 20/30; C12Q 1/6886; G01N 33/50; G16H 20/10; G16H 50/20

(52) U.S. Cl. CPC G16C 20/30; C12Q 1/6886; G01N 33/5011; G16H 20/10; G16H 50/20; C12Q 2600/156

(57) ABSTRACT

The embodiment discloses an optical density measurement and testing including a purification device that separates live cancer cells from dead and non-cancer cells of a microbiology sample. A drug addition device and a drug dosage sequencer introduce controlled dosages of at least one drug treatment into the live cancer cells. An optical density measurement device with an optical sensor captures high-resolution images of the live cells at predetermined intervals, and an optical spectrophotometric reader quantifies cell populations following treatment. A flow cytometry device measures fluorescent intensities of immune checkpoint markers and tumor antigens to generate immune system activation profiles. A processor subsystem analyzes cell death rates and immune responses to create integrated drug response profiles, and a computer application compares these results with patient-specific genetic and clinical data to produce personalized treatment recommendations.

### BACKGROUND

Testing of products and materials is performed in most industries and professions. Measurements are the key to providing the results of the testing. What size, shape, temperature, viscosity, and other factors are needed to determine the outcome for testing results and verify whether the products and materials meet the initial design criteria. To perform accurate measurements, a variety of devices and systems are required, depending on the nature of the testing and the physical environment. Many measurement devices and systems are unknown for testing, which could be a good match for the testing and provide more accurate results than previously understood.

### BRIEF SUMMARY OF THE INVENTION

The present invention is an optical density measurement and testing device configured to evaluate and personalize drug treatments for cancer therapy by directly testing purified live cancer cells derived from a patient sample. In one embodiment, the system includes a purification device that receives a microbiology sample, such as a biopsy specimen, and distinguishes and separates live cancer cells from dead cancer cells and non-cancerous cells. The purified live cancer cells are deposited into at least one microplate that serves as the culture environment for downstream testing. A drug addition device, operably coupled to the microplate, introduces one or more candidate drug treatments into the purified cancer cells. A drug dosage sequencer device controls the administration of different dosages of the treatment drugs, including sub-therapeutic and supra-therapeutic concentrations, to permit comparative evaluation of drug activity across a range of conditions.

In one embodiment, an optical density measurement device equipped with a high-resolution optical sensor captures sequential images of the cancer cells at predetermined intervals, thereby enabling quantification of live cell populations over time. An optical spectrophotometric reader is coupled to the optical density measurement device to measure concentration and growth of the live cells following drug administration. A flow cytometry subsystem is further integrated to measure fluorescent intensities of immune checkpoint markers and tumor antigens, including PD-1, PD-L1, and CTLA-4, expressed in the cancer cells. These measurements are processed to quantify levels of immune antigen expression and to generate dynamic immune activation profiles induced by drug treatment.

In one embodiment, a processor subsystem of a computer analyzes the sequential optical and cytometric measurements to determine rates of live cell death, changes in cell population growth, and immune antigen stimulation and release. The integrated analysis produces multidimensional profiles of drug response and immune system activation unique to the patient's cells. A computer application, coupled to the processors, compares these integrated profiles to known patient-specific information, including genetic markers, previously identified drug resistances, and known allergies or intolerances to candidate treatments. The application generates interactive treatment options for clinician review that balance therapeutic efficacy with patient tolerability, thereby enabling the development of personalized drug treatment recommendations optimized for the individual patient rather than relying solely on population-based treatment guidelines.

In another embodiment, the drug delivery device is used to add a single or a combination of drug treatments to the purified microbiology sample, live cancer cells at concentrations consistent with current NCCN chemotherapeutic guidelines. The optical density device is used to capture optical, photometric, and fluorescent images of the microbiology sample and live cancer cells at different predetermined intervals. At the first interval, the baseline image is captured before the addition of any NCCN-approved drug treatment or treatments to establish a baseline image for comparative purposes during the time course. Additional photos are captured at subsequent intervals after infusion of at least one drug treatment. The subsequent interval captured images are used to measure a change in cell density and fluorescent intensity of cell death markers including Annexin-V, caspases, and other markers of cell death caused by the drug treatments.

The changes in cell density and fluorescent measurements are used in a process to determine the death rate of the live cancer cells in the microbiology sample caused by the treatment drugs. The death rate determination is processed with a computer coupled to an optical density and fluorescent detection device. The interval captured images are recorded and measured to determine the population of the microbiology sample containing live cancer cells at the different predetermined intervals. In one embodiment, at least one computer having an optical density application wirelessly coupled to the computer is used to measure the changes in the microbiology sample population of living cells using the captured images to determine measured rates of death of the living cells over a period.

### BRIEF DESCRIPTION OF THE DRAWINGS

FIG. 1 shows, for illustrative purposes only, an example of an optical density measuring and testing device system of one embodiment.

FIG. 2 shows, for illustrative purposes only, an example of an optical density measurement and testing device results of one embodiment.

FIG. 3 shows, for illustrative purposes only, an example of an overview of a method and devices for direct apoptosis assay of purified cells of one embodiment.

FIG. 4 shows, for illustrative purposes only, an example of a cancer companion diagnostic for chemotherapy in one embodiment.

FIG. 5 shows, for illustrative purposes only, an example of performing a cancer companion diagnostic direct apoptosis assay of purified cancer cells of one embodiment.

FIG. 6 shows a block diagram of an overview flow chart of receiving patient biopsy tissue samples in one embodiment.

FIG. 7 shows a block diagram of an overview flow chart of assaying apoptosis of purified cancer cells in the culture of one embodiment.

FIG. 8 shows a block diagram of an overview of a direct APOP assay of purified cells of one embodiment.

FIG. 9 shows a block diagram of an overview of the extended APOP assay decision tree of one embodiment.

FIG. 10 shows a block diagram of an overview of the pre-APOP assay decision tree of one embodiment.

FIG. 11 and the block diagram show an overview of the parallel APOP assay decision tree of one embodiment.

FIG. 12 shows a block diagram of an overview of the interpretation of APOP results for a series of drugs or combinations in one embodiment.

FIG. 13 shows a block diagram of an overview of situations of one embodiment.

FIG. 14 shows a block diagram of an overview of situations continued in one embodiment.

FIG. 15 and the block diagram show an overview of the interpretation of APOP results for drugs or combinations based on the amount of O.D. change in one embodiment.

FIG. 16 shows a block diagram of an overview of the interpretation of APOP results for drugs with similar mechanisms of action in one embodiment.

FIG. 17 shows a block diagram and an overview of the advanced interpretation of APOP results using O.D. change and maximum O.D. increase from a single drug or a combination of one embodiment.

FIG. 18 shows a block diagram of an overview of enhancing drug development decisions by use of the APOP assay and cell growth inhibition in one embodiment.

FIG. 19 shows a block diagram of an overview of a method to reduce the cost of chemotherapy and/or drug therapy for cancer in one embodiment.

FIG. 20 is a block diagram of an overview of a method to promote immune therapy effects of immuno-active drugs and/or immune cells in treating cancer or leukemia in one embodiment.

FIG. 21 shows a block diagram of an overview of cancer or leukemia cells of one embodiment.

FIG. 22 shows a block diagram of an overview of a method to evaluate whether to consider using immunoactive drugs to treat cancer in one embodiment.

FIG. 23 shows a block diagram of an overview of measuring immune markers before the APOP assay of one embodiment.

FIG. 24 shows a block diagram of an overview of the APOP assay cancer cells of one embodiment.

FIG. 25 shows a block diagram of an overview of a method to identify the non-equivalences of drugs in one embodiment.

FIG. 26 shows a block diagram of an overview of using the APOP assay of one embodiment.

FIG. 27 shows a block diagram of an overview flow chart of a method for identifying an anti-apoptosis drug of one embodiment.

FIG. 28 shows, for illustrative purposes only, an example of a direct APOP assay of purified cells application of one embodiment.

FIG. 29 shows, for illustrative purposes only, an example of the measuring and testing results platform of one embodiment.

FIG. 30 shows a block diagram of an overview flow chart of programmed cell death overcoming apoptosis resistance in one embodiment.

FIG. 31 shows a block diagram of an overview flow chart of somatic gene variants of one embodiment.

FIG. 32 shows, for illustrative purposes only, an example of personalized chemotherapy drug dosing of one embodiment.

FIG. 33 shows, for illustrative purposes only, an example of personalized chemotherapy drug dosing of one embodiment.

FIG. 34 shows a block diagram of an overview of the APOP assay for active tumor cell death in one embodiment.

FIG. 35 shows a block diagram of an overview flow chart of a method for quantitatively tracking neoplastic progression in a subject of one embodiment.

FIG. 36 shows a block diagram of an overview of relative and absolute cancer-cell death metrics of one embodiment.

### DETAILED DESCRIPTION OF THE INVENTION

In the following description, reference is made to the accompanying drawings, which form a part hereof, and which are shown by way of illustration a specific example in which the invention may be practiced. It is to be understood that other embodiments may be utilized, and structural changes may be made without departing from the scope of the present invention.

The embodiments disclose an optical density and fluorescent measurement and testing system that includes a purification device configured to detect and isolate live cancer cells in a patient microbiology sample. A drug delivery device 116 is used to infuse a drug treatment into live cancer cells. An optical density and fluorescent imaging device are used to capture images of the microbiology samples. An optical microplate spectrophotometric reader couped to the optical density device is used to measure the density of living cells after infusion of at least one drug treatment, and a fluorescent imager is used to quantify and identify cells that are expressing markers of cell death, annexin V, and caspases. A computer coupled to the optical microplate spectrophotometric reader is used to determine measured rates of death over a period based on the interval population growth data. An optical density application operating on the computer is used to process measured rates of death and known predetermined genetic markers, resistances, and allergies of the patient associated with the drug to generate clinician treatment recommendations for the patient.

For the optical density measuring and testing device of the present invention, it should be noted that the descriptions that follow, for example, the term APOP is used to describe apoptosis and is related to direct death assays of purified cells. The descriptions that follow, referring to APOP and APOP assays, are for illustrative purposes, and the underlying system can apply to any number and multiple types of medical drug treatments and systems. In one embodiment of the present invention, the systems and devices used for direct APOP assay of purified cells can be configured using several drugs for testing. The devices for direct APOP assay of purified cells may be configured to include several cell purification technologies and several next-generation sequencing technologies using the present invention.

The term “apoptosis” used herein refers to a genetically directed process of cell self-destruction that is marked by the fragmentation of nuclear DNA, activated either by the presence of a stimulus or removal of a suppressing agent or stimulus, a normal physiological process eliminating DNA-damaged, superfluous, or unwanted cells, and when halted (as by genetic mutation) may result in uncontrolled cell growth and tumor formation and additionally is expressed without any change in meaning as “APOP” in any case lower, upper or mixed. The optical density measurement device includes an optical microplate spectrophotometric reader used to measure the density of living cells.

The fluorescent microplate reader is used to measure and quantify markers of cell death (apoptosis, pyroptosis, methuosis, cuproptosis, ferroptosis, necroptosis, and other defined mechanisms of cell death) in living cells. The term “O.D.” used herein refers to the term “Optical Density” and is expressed without any change in meaning as “optical density” in any case, lower, upper, or mixed. The term “APOP” used herein refers to an assay to test and measure the apoptosis and cell death effectiveness of a single drug or combination of drugs against purified cells, including cancer cells. The term “companion diagnostic” used herein refers to a diagnostic test used as a companion to a therapeutic drug to determine its applicability to a specific person. The term “antigen” used herein refers to a protein, toxin, or other foreign substance that induces an immune response in the body, especially the production of antibodies or a cellular response. The term “Immunotherapy” used herein refers to a treatment to stimulate or restore the ability of the immune (defense) system to fight infection, cancer, and disease. The term “cannabinoid” used herein refers to any chemical in marijuana that causes drug-like effects throughout the body, including the central nervous system and the immune system. The term “CBD” used herein refers to legal nonintoxicating cannabinoids found in cannabis and hemp.

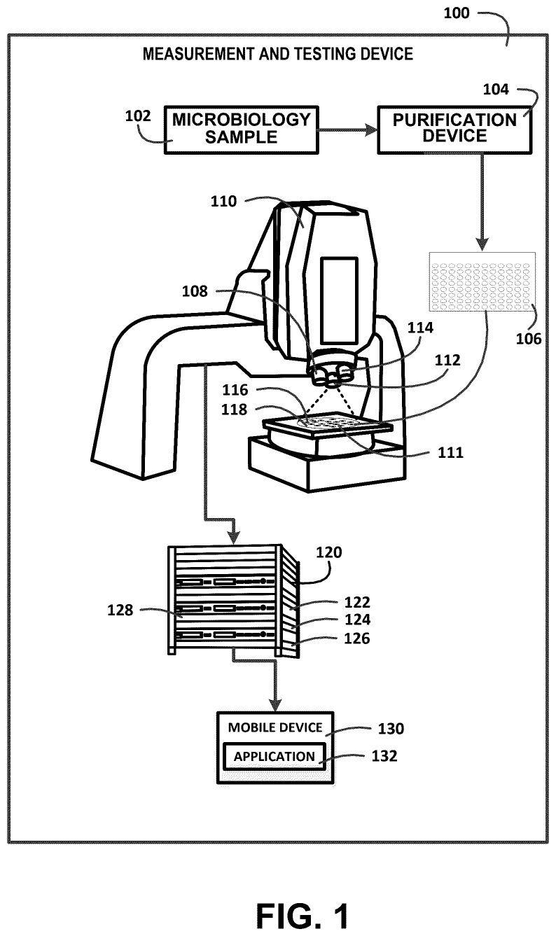

FIG. 1 shows, for illustrative purposes only, an example of an optical density and fluorescent measuring and testing device system of one embodiment. FIG. 1 shows a measurement and testing device 100 used for drug treatment testing on a patient tissue microbiology sample 102. The patient tissue microbiology sample 102 is processed in a purification device 104 to detect live cancer cells and sorts those from dead cells and other cells and isolates the live cancer cells. The live cancer cells are deposited into microplate 106 wells in a liquid. A drug delivery device 116 infuses at least one drug treatment at specific dosages measured with a drug dosage sequencer device 118 at predetermined time intervals. An optical microplate spectrophotometric reader 110 captures high-resolution images using an illumination source 112 and optical sensor 114, of the cancer cells in the microplate 106 wells. An optical density measurement device 111 measures each cancer cell to detect changes in the cell diameter and other characteristics that indicate whether the cancer cell is affected by the infusion of the drug treatment. A flow cytometry device 108 illuminates the cancer cells with fluorescent light to measure the fluorescent intensity in the cell to measure life signs and determine signs of apoptosis.

The captured images, optical density measurement device 111 measurements and flow cytometry device 108 fluorescent intensity determinations are transmitted to a remote server 120. The server 120 automatically processes the data through an artificial intelligence 122 system having machine learning 124 and at least one set of computer-readable instructions 126. The artificial intelligence 122 system having machine learning 124 analyzes the data to calculate the treatment results and make evaluations of the efficacy of the drug treatment in killing the cancer cells. The artificial intelligence 122 system having machine learning 124 further compares the results of one drug treatment versus a different drug treatment and dosage level personalized for the specific patient. The artificial intelligence 122 system having machine learning 124 generates suggested treatment regimens for the patient cancer treatment and transmits the testing results and makes recommendations to the patient care provider through the digital clinician interface mobile device 130 with an application 132.

In one embodiment, the purification device 104 for cancer cell purification may be using fluorescence cell sorting, magnetic cell sorting, and buoyancy cell sorting that is used to detect and isolate live cancer cells from dead cancer cells and non-cancer cells of a microbiology sample 102. In certain embodiments, the purification device 104 includes a cell isolation system configured to obtain a population of live cancer cells from a patient-derived microbiology sample 102. The cell isolation system may operate by distinguishing live cancer cells from dead cells and non-cancer cells based on physical, chemical, optical, or biological characteristics. Such systems may employ, without limitation, detection of cell morphology, optical properties, fluorescence signals, magnetic or antibody-based binding, microfluidic separation, or other equivalent mechanisms that enable identification and separation of live cancer cells. Any purification approach that yields a sufficiently enriched population of live cancer cells for subsequent analysis may be employed. In all cases, the isolated live cancer cells are collected into at least one vessel, such as a microplate 106, to permit downstream drug treatment, imaging, and measurement.

At least one microplate 106 coupled to the purification device 104 is used to contain the microbiology sample 102 of purified microbiological living cells in a liquid. A plurality of microplate 106 well images is a plurality of “wells” to include cell cultures within a well, which is described as a small test tube. A drug delivery device 116 coupled to at least one microplate 106 is used to infuse at least one drug treatment into the purified microbiology sample 102, live cancer cells. A drug dosage sequencer device 118 coupled to the drug delivery device 116 is used to measure different treatment drug dosages to infuse at least one drug treatment into the purified microbiology sample 102 live cancer cells to gather results to determine a range of dosage efficacy over time. Dosage determination will follow NCCN guidelines with the addition of dosages above and below the NCCN guidelines to establish if a higher or lower dosage than is traditionally prescribed would be equally or more efficacious for the treatment of an individual patient. The goal of dosage determination (bracketing) is to maximize the cell death effect that is seen for a given medication in a specific patient with the minimum effective dosage to effect cell death.

A measurement and testing device 100 including an optical density measurement device 111 coupled to an optical density measurement activation cycling device and an optical microplate spectrophotometric reader 110. In one embodiment, the optical density measurement device 111 is used to determine the concentration of microbiological cells in a liquid culture of a microbiology sample 102. The optical density measurement device 111 captures an image of the microbiology sample 102 for an optical density measurement process of the image captured. The optical density measurement device 111 captures a high-resolution image using an optical sensor coupled to it. In this example, an interval no. The captured image of 3 optical density microbiology sample 102 are stored in server 120, having at least one digital processor 128, at least one communication device 124, an artificial intelligence 122 device with integrated machine learning 124 functions. The artificial intelligence 122 device with integrated machine learning 124 functions is implemented using at least one set of computer-readable instructions 126.

The server 120 provides the interval number. 3 optical density microbiology sample 102 captured image of a microplate well image 242 to a computer to measure the concentration of microbiology cells in the determined area of the microplate 106 well containing the microbiology sample 102. Coupled to the server 120 is an artificial intelligence 122 device with integrated machine learning 124 functions, which are implemented using at least one set of computer-readable instructions 126. The concentration is determined by the microbiology cell count of individual cells identified by the optical sensor. The physical area of the microplate 106-well is a predetermined area. A flow cytometry device 108 is used for fluorescent measurement. In one embodiment the flow cytometry device 108 is used to analyze and sort individual cells suspended in a fluid stream by passing them single-file through a laser beam. As each cell passes through, detectors measure scattered light and fluorescence emitted from labeled markers, providing information about cell size, granularity, and the presence of specific proteins or biomarkers. This allows rapid, quantitative, classifiable, and to isolate distinct cell populations for diagnostic or therapeutic applications. An optical density measurement activation cycling device coupled to the optical density device is used to activate the optical density image capture process at predetermined intervals.

The results are represented as an average number of microbiology cells in a predetermined area, for example, a square inch or a square centimeter. This result is a base concentration at the time the optical density measurement image capture is captured. The determination of concentration based on changes in optical density measurements and subsequent analytical evaluations serves as the foundation for generating recommendations to assist a clinician in formulating a treatment plan of one embodiment.

In one embodiment, a purification device 104 is coupled to a disruption device to isolate viable cancer cells from a dissociated tissue microbiology sample 102 for downstream analysis. The purification device 104 functions using techniques including fluorescence cell sorting, magnetic bead separation, or buoyancy-based fractionation to selectively separate live cancer cells from dead cells and non-cancerous components. The coupling ensures that the single-cell suspension generated by the disruption device flows directly into the purification device 104 under sterile conditions, minimizing microbiology sample 102 degradation and variability. The purified cancer cells are then deposited into at least one microplate 106 for culture, drug testing, and optical density measurement. By producing a high-yield, enriched population of living cancer cells, the purification device 104 establishes the foundation for accurate apoptosis assays, dosage-response studies, and machine learning 124 analysis of patient-specific drug effectiveness.

In one embodiment, a disruption device is coupled to a purification device 104 to mechanically and enzymatically dissociate a patient-derived cancer biopsy into a viable single-cell suspension for downstream analysis. The disruption device functions by applying controlled mechanical agitation and optional enzymatic digestion to break down solid tissue of the microbiology sample 102 into separated living cells while preserving their viability. This dissociated cell suspension is directly transferred to the purification device 104, which isolates cancer cells from non-cancerous or dead cells for subsequent testing. The coupling between the disruption device and the purification device 104 ensures that the generated cell suspension is efficiently delivered in a sterile and reproducible manner, minimizing operator variability and maintaining microbiology sample 102 integrity for accurate optical density measurements, apoptosis assays, and drug treatment evaluations.

In one embodiment, a treatment matrix and incubation device are coupled to at least one microplate 106 to deliver chemotherapeutic agents into purified cancer cell cultures and maintain them under controlled growth conditions. The treatment matrix defines which wells of the microplate 106 receive specific drug treatments or combinations, generating a detailed plate map of exposure conditions, while the incubation device maintains the microplate 106 at 37° C. with 5% CO₂ and regulated humidity to replicate physiological conditions. This coupling ensures that drug infusion from the treatment matrix is precisely aligned with incubation, so that cancer cells remain metabolically active and responsive throughout the drug testing period. By integrating liquid handling with environmental control, the treatment matrix and incubation device standardize drug delivery, sustain cell viability, and enable reproducible high-throughput testing across multiple microplate 106 wells for downstream apoptosis assays and optical density measurements.

In one embodiment, a microplate 106 is coupled to a purification device 104 to contain purified microbiological living cells in liquid suspension for subsequent testing. The microplate 106 comprises a plurality of wells, each functioning as a miniature test tube, configured to hold discrete cultures of purified cancer cells in defined media. The coupling ensures that purified cells generated by the purification device 104 are deposited directly into the wells under sterile conditions, forming a uniform and replicable assay environment. The microplate 106 is further coupled to an optical density measurement device 111, which aligns its optical path with each well to capture transmitted or scattered light for density analysis, and to a drug delivery device 116 for the infusion of chemotherapeutic agents into designated wells. By serving as the central containment platform, the microplate 106 provides consistent optical geometry, supports parallel analysis across multiple conditions, and maintains microbiology sample 102 stability for downstream incubation, imaging, and drug response evaluations.

In one embodiment, a drug delivery device 116 is coupled to at least one microplate 106 to infuse a controlled amount of chemotherapeutic agents into purified microbiology live cancer cells. The drug delivery device 116 is configured as an automated liquid handling instrument that aspirates sterile drug solutions from reservoirs and dispenses precise microliter volumes into designated wells of the microplate 106 according to a programmed treatment matrix. The coupling ensures that the infused drugs directly contact the viable cancer cells contained within each microplate 106 well, enabling standardized exposure across multiple experimental conditions. The device functions under sterile conditions to prevent contamination and is integrated with sequencing protocols to deliver single drugs or combinations at concentrations consistent with NCCN guidelines, including dosages above and below standard ranges for bracketing efficacy. By operating in tandem with the microplate 106 and incubation device, the drug delivery device 116 ensures reproducibility, supports high-throughput testing, and provides reliable input for downstream optical density measurements, fluorescence imaging, and apoptosis assay evaluations.

In one embodiment, a drug dosage sequencer device 118 is coupled to the drug delivery device 116 to regulate the timing and concentration of chemotherapeutic infusions into purified cancer cell cultures. The drug dosage sequencer device 118 operates as an automated control system that schedules delivery of drugs in microliter volumes into specific microplate 106 wells at predetermined intervals, consistent with NCCN chemotherapeutic guidelines and extended dosages above and below the guidelines for efficacy bracketing. The coupling ensures that the sequencer directs the drug delivery device 116 to administer single drugs or combinations in varying concentrations over defined time courses, allowing precise titration of dosage-response relationships. This integration maintains sterile conditions while cells remain incubated at 37° C. with CO₂ regulation, enabling real-time monitoring of treatment effects. By providing sequential control of dose levels and timing, the drug dosage sequencer device 118 enables systematic testing of dosage ranges, identification of minimum effective concentrations, and generation of personalized treatment data that are later analyzed through optical density measurements, fluorescence imaging, and Al-assisted evaluations.

In one embodiment, a detailed plate map is coupled to the treatment matrix and incubation device to define and track the specific drug treatments assigned to each microplate 106 well. The detailed plate map functions as a digital or physical layout that records which chemotherapeutic agents, combinations, or dosage levels are dispensed by the treatment matrix into designated wells of the microplate 106. The coupling ensures that the incubation device maintains the treated wells under uniform conditions while the plate map preserves the experimental schema for accurate downstream interpretation. Each entry in the plate map corresponds to a precise well location, creating a structured record that links drug identity, dosage, and timing to observed cellular responses. By aligning the treatment matrix operations with incubation, optical density measurement, and fluorescence imaging, the detailed plate map provides a reference framework for correlating drug exposure to cell viability, apoptosis kinetics, and biomarker expression, thereby ensuring reproducibility and enabling high-throughput comparative analysis.

In one embodiment, an optical density measurement device 111 is coupled to at least one microplate 106 to quantify the concentration and viability of purified microbiology live cancer cells during drug testing. The optical density measurement device 111 includes an optical microplate spectrophotometric reader 110 coupled to an illumination source 112 and optical sensor 114 to capture high-resolution images using an illumination source 112 and optical sensor 114 optical sensor 114 and to capture high-resolution images and measure light absorbance or scatter across microplate 106 wells containing cell suspensions. The coupling ensures that transmitted light passes through each microplate 106 well, allowing the device to calculate changes in optical density that correspond to cell growth, metabolic activity, or apoptosis following drug infusion. The optical density measurement device 111 is further coupled to an optical density measurement activation cycling device to trigger imaging at predetermined intervals, generating temporal datasets of population changes. By providing both numerical absorbance values and image-based data, the optical density measurement device 111 delivers a non-destructive, repeatable method to monitor live-cell dynamics under treatment conditions, forming a foundation for analysis modules, artificial intelligence 122 processing, and clinician decision support.

In one embodiment, an image and fluorescent measurement device is coupled to at least one microplate 106 to capture high-resolution optical images and quantify fluorescent signals from purified microbiology live cancer cells during treatment. The image and fluorescent measurement device functions as a multimode microplate reader and imager, configured to record transmitted light, scattered light, and fluorescence intensity from dyes or probes that indicate apoptosis including Annexin-V or caspases. The coupling ensures that each microplate 106 well is illuminated and imaged under uniform optical paths, while fluorescence emissions from drug-treated cells are measured and digitally recorded for analysis. The device operates in tandem with the optical density measurement device 111 to provide complementary datasets, absorbance values indicating cell density and fluorescent intensities indicating programmed cell death mechanisms. By integrating imaging and fluorescence quantification, the image and fluorescent measurement device enables detailed kinetic monitoring of apoptosis, drug-induced cytotoxicity, and morphological changes across multiple wells, providing a multiparametric dataset for downstream analysis modules and AI-assisted interpretation.

In one embodiment, at least one analysis module is coupled to the optical density measurement device 111 and the image and fluorescent measurement device to process collected cell growth and apoptosis data. The analysis module receives high-resolution images, absorbance readings, and fluorescent intensity measurements from the coupled devices, and applies computational routines including segmentation, pattern recognition, and statistical modeling. The coupling ensures that optical density and fluorescence signals are synchronized with temporal and spatial identifiers from the microplate 106, allowing accurate correlation of drug dosage, treatment timing, and observed cellular responses. The analysis module calculates cell concentration, tracks morphological changes, quantifies death marker expression, and generates growth curves over predetermined intervals. By integrating these inputs, the analysis module produces standardized datasets and preliminary interpretations that can be transmitted to an artificial intelligence 122 system with machine learning 124 for deeper evaluation and the generation of personalized treatment recommendations.

In one embodiment, an artificial intelligence 122 system having machine learning 124 is coupled to at least one analysis module to evaluate multiparametric datasets from optical density, fluorescent imaging, and biomarker measurements. The artificial intelligence 122 system receives processed outputs including cell viability curves, apoptosis kinetics, genetic marker associations, and drug dosage response profiles, and applies machine learning 124 algorithms trained on large-scale datasets to recognize apoptosis patterns and predict treatment outcomes. The coupling ensures that the analysis module provides structured data directly to the AI system, which integrates optical, fluorescent, genomic, and temporal variables to generate an “onco-death score” ranking drug effectiveness. The AI system functions to reduce the time and complexity of manual interpretation by clinicians, standardize evaluation across thousands of images and measurements, and generate actionable treatment recommendations. Through iterative learning, the artificial intelligence 122 system with machine learning 124 improves predictive accuracy and adapts to new drug-response patterns, forming the decision-making backbone of the personalized cancer therapy workflow.

In one embodiment, at least one analytical device is coupled to the analysis module to detect, identify, and monitor soluble cancer markers associated with treatment response. The analytical device functions by sampling microplate 106 well contents or associated culture media to measure biochemical indicators including VEGF, sPD-L1, MMP9, Ki67, CEA, AFP, B-HCG, CA15-3, CA19-9, CA27.29, and CA125. The coupling ensures that marker detection is integrated with optical density and fluorescent imaging data, allowing direct correlation of soluble biomarker expression with measured cell viability and apoptosis events. The analytical device transmits marker concentration data back to the analysis module, where it is combined with growth curves, fluorescence intensity changes, and treatment matrix information. This integration provides a comprehensive profile of cellular and molecular responses, enabling both cross-validation of optical measurements and enrichment of the dataset used by the artificial intelligence 122 system for generating personalized therapeutic recommendations.

In one embodiment, a digital clinician interface is coupled to the artificial intelligence 122 system having machine learning 124 to display treatment results, interpretations, and suggested clinical decisions. The digital clinician interface functions as a secure application installed on a clinician's mobile device 130 with an application 132 including a computer, tablet, or smartphone, configured to receive AI-processed outputs including onco-death scores, drug efficacy rankings, apoptosis kinetics, genetic marker correlations, and dosage-response profiles. The coupling ensures that processed data from the AI system is transmitted in real time to the clinician interface, where it is formatted into structured reports, graphs, and decision trees. The interface allows clinicians to review personalized treatment recommendations alongside comparative drug effectiveness and biomarker analysis, and to discuss the results directly with patients. By providing intuitive visualization, real-time access, and integration with patient-specific datasets, the digital clinician interface enables evidence-based therapeutic planning and supports clinical decision-making at the point of care.

The optical density measurement device 111 is coupled to an optical sensor and to a microplate 106 to provide image-based analysis of at least one microbiology sample 102. The device captures high-resolution images of purified microbiology live cancer cells suspended in liquid contained in the wells of the microplate 106. The measurement device calculates optical density by recording transmitted or scattered light data, while the imaging capability provides information on changes in cell morphology and population density over time. The combination of density measurement and imaging functions enables quantitative analysis of microbiology sample 102 growth during defined monitoring periods.

The microplate 106 holds purified microbiology live cancer cells in liquid media within multiple wells, with each well functioning as an independent analysis environment. The microplate 106 provides a uniform structure that ensures consistent optical paths for imaging and measurement. The coupling of the optical density measurement device 111 to the microplate 106 aligns the optical path of the sensor with each microbiology sample 102 well, allowing repeatable and accurate measurements across the plate. Parallel analysis of multiple wells is achieved by cycling the device across the microplate 106 structure, producing consistent datasets from identical containment environments.

The optical sensor captures the light transmitted through the microbiology microbiology sample 102 and converts this optical information into digital image data. The optical sensor 114 records high-resolution images of the purified microbiology live cancer cells and provides a dataset for calculating changes in optical density. The sensor is configured to capture data at defined time points, enabling sequential monitoring of growth and metabolic activity. The imaging function provides detail on both cell concentration and morphological features that can be correlated with measured optical density.

The predetermined intervals define the time points at which images are captured and measurements are performed. These intervals may be programmed through system control logic or dynamically adjusted by processing routines based on prior readings. The intervals provide temporal datasets that track the progression of microbiology sample 102 growth. Consistent image capture and measurement at these defined time points enable monitoring of changes in population density, media turbidity, and related biological interactions throughout the experiment.

The sensor array is configured to monitor biological and environmental parameters associated with the microbiology sample 102. Each sensor in the array provides a dedicated function, including detection of temperature, pH, dissolved oxygen, or chemical composition of the liquid medium. The sensors generate electrical signals representative of their respective parameters, and these signals are transmitted to the system's processing module. The use of multiple sensors in a unified array allows for comprehensive tracking of microbiology sample 102 conditions in real time. This enables correlation between optical density measurements, captured images, and environmental variables within the microplate 106.

The control logic governs operation of the optical density measurement device 111, the optical sensor, and the timing of predetermined intervals. The logic coordinates the initiation of image capture and measurement cycles, ensuring that each event occurs in sequence and without interference. Control functions include activating illumination sources, triggering the optical sensor, storing digital images, and recording optical density readings. The control logic may also adjust capture intervals or sensor gain settings based on prior measurements to optimize data quality. This provides a systematic and repeatable process for recording population growth of purified microbiology live cancer cells.

The data analysis module processes the high-resolution images and optical density readings obtained from the microplate 106 microbiology sample 102. The module applies computational routines to calculate population density, identify cell morphology changes, and detect growth patterns over time. Data from the sensor array is incorporated into these calculations, allowing environmental conditions to be associated with observed biological responses. The analysis routines may include segmentation, pattern recognition, and statistical models configured to measure differences between consecutive time points. The module provides outputs in the form of growth curves, concentration values, and predictive indicators of metabolic activity.

The wireless communication interface provides connectivity between the measurement system and external computing devices. The interface transmits captured image data, optical density readings, and sensor array values to a remote mobile device 130 with an application 132 including a mobile phone, tablet, or computer system. The wireless communication interface is configured to operate using common standards including Bluetooth® or Wi-Fi, allowing flexible integration with laboratory information management systems or cloud-based analysis platforms. Data transmission occurs automatically following each predetermined interval, ensuring that remote devices maintain updated datasets for review or further processing.

The processing unit receives signals from the optical sensor, the sensor array, and the control logic to execute measurement and analysis tasks. It is configured to process captured high-resolution images, convert raw optical density data into usable numerical values, and combine these with environmental readings from the sensor array. The processing unit applies programmed algorithms to detect changes in microbiology cell populations across successive predetermined intervals. These algorithms include digital filtering, pattern recognition, and data correlation methods. The processing unit also directs instructions back to the control logic to adjust measurement cycles or parameters as needed for optimal data acquisition.

The storage medium is configured to retain the data generated by the optical density measurement device 111, the optical sensor, and the processing unit. Stored content includes raw image files, processed numerical results, sensor values, and computed growth models. The storage medium may consist of local memory integrated within the measurement system or a removable module to allow transfer of large datasets. Data is organized according to time stamps, microbiology sample 102 identifiers, and measurement intervals to maintain a consistent and traceable record. This provides long-term accessibility for retrospective analysis, validation of results, and regulatory compliance.

The power source supplies electrical energy to the optical density measurement device 111, the optical sensor, the processing unit, and the wireless communication interface. In one embodiment, the power source includes at least one rechargeable battery configured to provide continuous operation across multiple measurement cycles. The battery may be recharged through a connection to an external power input port. The power source is also configured with voltage regulation to ensure stable operation of sensitive components including the optical sensor and data analysis module. At least one analytical device is coupled remotely to the at least one analysis module configured to detect, identify, and monitor soluble cancer markers. The presence of a rechargeable power source allows portable and autonomous deployment of the measurement system in laboratory or field environments.

The illumination source is configured to provide consistent and controlled light exposure to the microplate 106. Light emitted by the illumination source passes through the liquid contained in the microplate 106 wells and interacts with the microbiology live cancer cells under observation. The transmitted or scattered light is then captured by the optical sensor, enabling calculation of optical density. The illumination source is designed to minimize spectral distortion and to deliver a uniform intensity across the surface of the microplate 106. This uniform illumination ensures that all wells receive comparable conditions, supporting accurate and repeatable measurements across the entire plate.

The microplate interface provides a physical and functional connection between the microplate 106 and the optical density measurement device 111. The interface includes a mechanical holder configured to align the microplate 106 with the optical sensor and illumination source. It ensures that the microplate 106 remains stationary during measurement cycles, preventing artifacts caused by vibration or movement. The microplate interface may also include thermal regulation elements to maintain a controlled temperature environment for the microbiology sample 102. By stabilizing the position and condition of the microplate 106, the interface enables consistent capture of high-resolution images and accurate optical density readings.

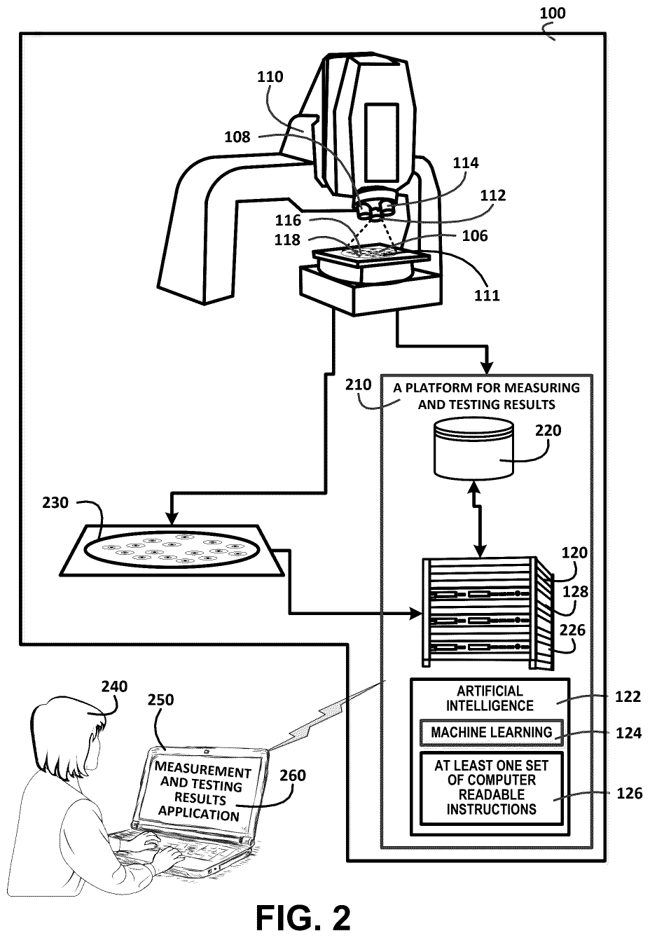

FIG. 2 shows, for illustrative purposes only, an example of optical density and fluorescent measurement and testing device results of one embodiment. FIG. 2 shows the measurement and testing devices 100, including the optical density measurement device 111, which can include an optical microplate spectrophotometric reader 110.

A drug delivery device 122 infuses at least one drug treatment at specific dosages measured with a drug dosage sequencer device 123 at predetermined time intervals.

At least one testing device, including the optical density measurement device 111, is used for optical density measurement image captures using the optical microplate spectrophotometric reader 110, optical sensor 114, illumination source 112, to capture at least one high resolution image 230 of at least one microbiology sample 102 of FIG. 1 in the microplate 106. The optical density of at least one microbiology sample 102 of FIG. 1 is measured using a computer 250 having an application 260 used by the clinician 240. Wherein the computer 250 is remotely coupled to the server 120 having at least one database 220, a plurality of processors 128, communication devices 226, artificial intelligence 122, machine learning 124 with at least one set of computer readable instructions 126. The changes in cell density measurements are used in a process to determine the death rate of the live cancer cells in the microbiology sample 102 caused by the treatment drugs.

A platform for measuring and testing results recorded on at least one server 120 includes an artificial intelligence 122 having machine learning 124 and at least one set of computer-readable instructions 126. The death rate determination is processed with a computer 270 coupled to the server 120, to access the microplate well image 242 data stored in the server 120 and to perform the concentration determination, an artificial intelligence 122 device with integrated machine learning 124 functions. The artificial intelligence 122 device with integrated machine learning 124 functions is implemented using at least one set of computer-readable instructions 126.

The interval captured images are recorded and measured to determine the population of live cancer cells in the microbiology sample 102 at the different predetermined intervals. In one embodiment, at least one computer with optical density and fluorescent applications is wirelessly coupled to the server 120. This computer is used to measure changes in the microbiology sample 102 population of living cells, using captured images to determine the measured rates of death of the living cells over a period. The determination results of the measured rates of death of the living cells over a period are stored in a measurement and testing database 220 coupled to the server 120.

The optical density measurement image capture is activated in a controlled environment of 37 degrees C. and 5% CO₂ conducive to cell growth. An optical density measurement activation cycling device coupled to the optical density device is used to activate the optical density image capture process at predetermined intervals. The optical density measurement image capture is activated at predetermined time intervals to measure changes in the population of the microbiology cells for a determination of the concentration level. The measurement activation cycle is first performed using the optical density measurement device 111 to measure microbiological cell growth without any treatment to measure a baseline growth rate.

The drug delivery device 116 is used to infuse at least one drug treatment into the purified microbiology sample 102, live cancer cells and is used for testing of specific NCCN-approved protocols and treatment drugs added to separate individual microplate 106 cultures of the microbiology sample 102.

A drug delivery device 116 infuses at least one drug treatment at specific dosages measured with a drug dosage sequencer device 118 at predetermined time intervals.

A drug dosage sequencer device 118 of FIG. 1 coupled to the drug delivery device 116 is used to measure different treatment drug dosages according to established NCCN guidelines to infuse at least one drug treatment to the purified microbiology sample 102 live cancer cells to gather results to determine a range of dosage efficacy over time. An optical microplate spectrophotometric reader 111 captures high-resolution images of the cancer cells in the microplate 106 wells.

The treated microbiology sample 102 is tested using the optical density measurement device 111 and fluorescent plate reader device to determine whether the treatment causes apoptosis or cell death of the microbiology sample 102.

A drug dosage sequencer device 118 of FIG. 1 coupled to the drug delivery device 116 is used to measure different treatment drug dosages according to established NCCN guidelines to infuse at least one drug treatment to the purified microbiology sample 102 live cancer cells to gather results to determine a range of dosage efficacy over time. If sufficient cells are available for additional drug dosage refinement, dosages above and below the established NCCN will be administered to the microbiology sample 102 to precisely titrate the dosage to the individual patient. The measurement results of the optical density measurement device 111 captured images analysis provide the concentrations of both living microbiology cells and dead microbiology cells, which indicate the efficacy of the treatment over the predetermined time intervals.

The optical density measurement device 111 measurement analysis results include the percentages of microbiology cell deaths, the rates at which the population declines (if at all), what period of time is needed for the treatment to become effective, and how long the treatment effectiveness lasts in terms of time. In these examples, the optical density measurement device 111 measurement activation cycles can be repeated with varying dosages to gather results to determine a range of dosage efficacy over time.

Optical density measurement results of microbiology sample 102 images captured at different intervals are stored in a measurement and testing database 220. FIG. 2 shows an interval no. 1 optical density microbiology sample 102 captured image of a microbiology sample 102. The captured image from interval number 1 is measured to determine a live cell population. In this example, the interval no. 2 measured population of live cells shows a decrease compared to the interval no. 1 measured population of live cells. The optical density measurement results are stored in a measurement and testing database 220. A server 120 coupled to the measurement and testing database 220 performs a comparative analysis of changes in the comparative analysis results and sends the comparative analysis results to a computer 270 to display measurement and testing results app 280 to a clinician 290 for review on the treatment testing of one embodiment.

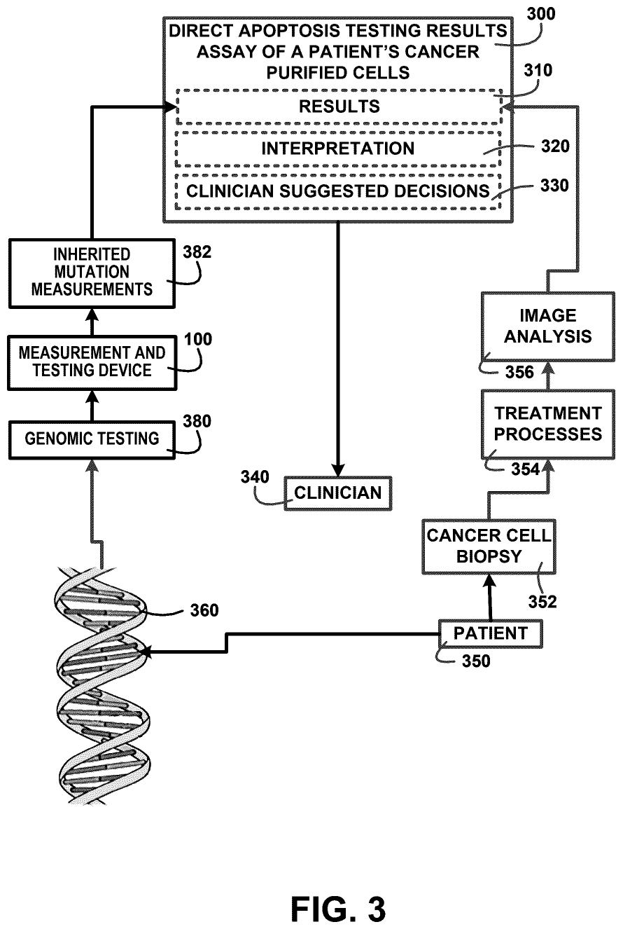

FIG. 3 shows, for illustrative purposes only, an example of an overview of a method and devices for direct apoptosis assay of purified cells of one embodiment. FIG. 3 shows a patient 350 providing a cancer cell biopsy 352 and a patient's DNA genomic testing sample 360. The method and devices for direct apoptosis assay of purified cells process the cancer cell biopsy 352 and DNA genomic testing 380 provided by the patient 350. The cancer cell biopsy 352 tissues are processed in at least one cell purification procedure. The purified cells are then processed in a series of apoptosis next-generation sequence testing treatment processes 354 with selected drugs and combinations of drugs to determine which is the most effective in killing image analysis 356, in this example, the patient's cancer cells. Recommending PARP inhibitors as part of suggestions to doctors includes using the APOP assay with and/or without next-generation sequencing, oral swabs, and/or blood in parallel to be able to assess where DNA mutations exist, for example, in a tumor or also due to bloodline mutations.

DNA genomic testing 380 is reviewed to identify genetic markers that show any variants in the genes that would affect the use of one or more drugs that could be used in a treatment regimen. Direct apoptosis testing results assay of a patient's cancer-purified cells 300 are correlated into results 310, interpretations 320, and clinician-suggested decisions 330. The results include measurement and testing device 100 used in genetic testing 380 to measure the genome for inherited mutation measurements 382. The correlated apoptosis testing results assay, including the results 310, interpretations 320, and clinician-suggested decisions 330, is transmitted, for example, to a clinician's mobile device 130 with an application 132. The clinician's mobile device 130 of FIG. 1 with an application 132 of FIG. 1 displays the apoptosis testing results using an apoptosis application installed on the clinician's mobile device 130 of FIG. 1 with an application 132 of FIG. 1. This allows the clinician 340 to review the results, interpretations, and suggested decisions with the patient 350 for planning a treatment course of one embodiment.

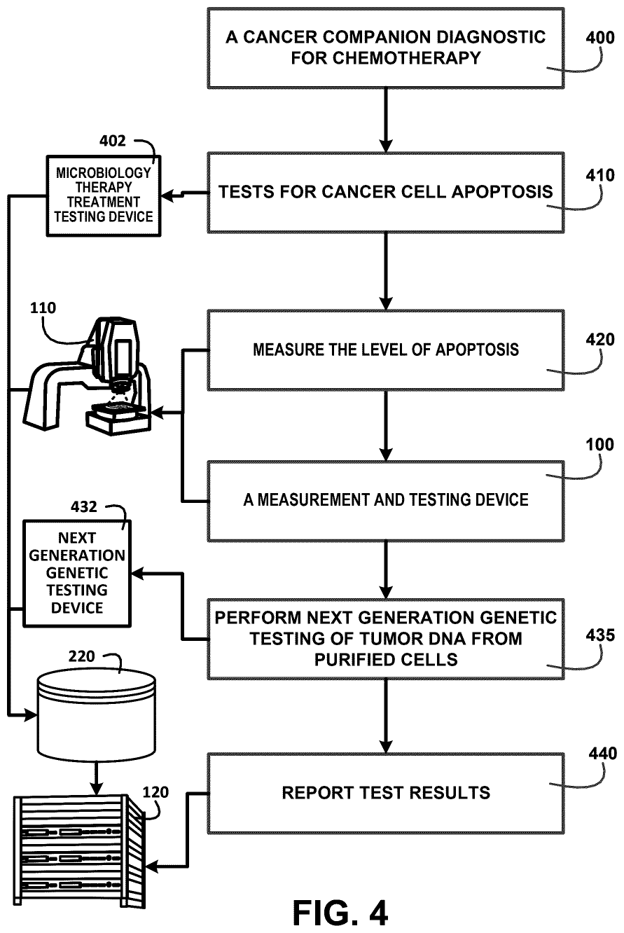

FIG. 4 shows for illustrative purposes only an example of a cancer companion diagnostic for chemotherapy of one embodiment. FIG. 4 shows a cancer companion diagnostic for chemotherapy 400 used to test for cancer cell apoptosis from a single chemotherapy drug alone, or in combination with other drugs or immunotherapy. Tests for cancer cell apoptosis 410 are performed using a microbiology therapy treatment testing device 402 with the optical density measurement device 111. To perform next-generation genetic testing of tumor DNA from purified cells 435 a next-generation genetic testing device 432 is used with the optical density measurement device 111.

The companion diagnostic data are stored in the measurement and testing database 220. The direct apoptosis testing results assay of a patient's cancer-purified cells 300 of FIG. 3 in one sequencing example of the results records a measure of the level of apoptosis 420 caused by the introduction of cannabinoids/CBD to cancer cells and stores data in the measurement and testing database 220 for processing in the server 120. Also, a measurement and testing device 100 measures the increase of immune antigen stimulation treatment to kill cancer cells and release antigens to the immune system, and stores data in the measurement and testing database 220 for processing in the server 120. Perform next-generation genetic testing on 435 tumor DNA samples from purified cells and genetic testing on healthy DNA from purified cells, and store data in the measurement and testing database 220 for processing on the server 120. The direct apoptosis testing results assay of a patient's cancer-purified cells 300 of FIG. 3 is used to report test results 440, interpretations, and suggested clinician decision trees electronically with a digital application to make the data available to clinicians for reviewing with patients from the server 120 of one embodiment.

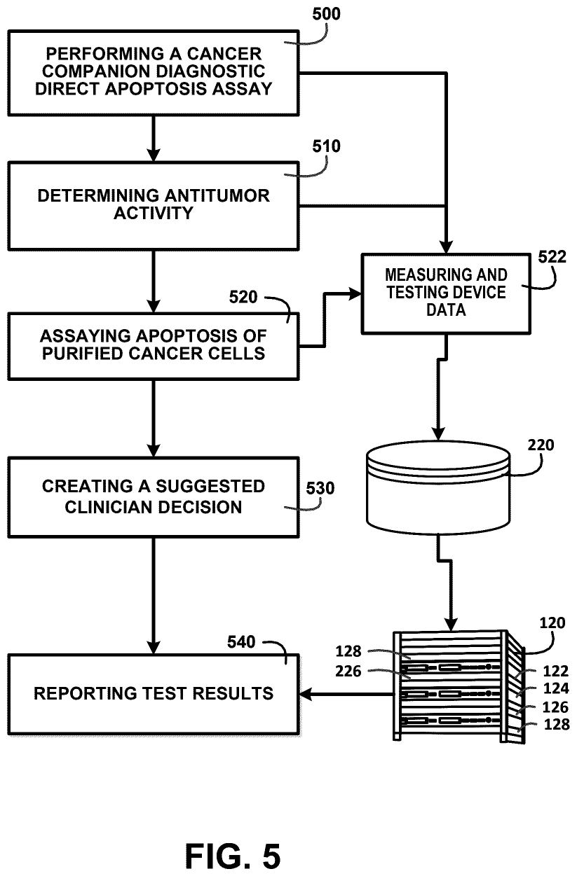

FIG. 5 shows, for illustrative purposes only, an example of performing a cancer companion diagnostic direct apoptosis assay of purified cancer cells of one embodiment. FIG. 5 shows performing a cancer companion diagnostic direct apoptosis assay 500 of purified cancer cells, with descriptions of processes shown in FIG. 5. After performing a cancer companion diagnostic direct apoptosis assay 500 of purified cancer cells, as shown in FIG. 5 are processes for determining antitumor activity 510 or other effects by growth inhibition or other methods. Processing continues with assaying apoptosis of purified cancer cells 520 in culture with descriptions of processes shown in FIG. 5. After performing the processes for assaying apoptosis of purified cancer cells in culture 520 as shown in FIG. 5 the processing continues for creating a suggested clinician decision 530 tree using the interpretations of the direct apoptosis assay of purified cancer cells results. Reporting test results 540, interpretations, and the suggested clinician decision tree with a digital application to a clinician's digital device. The processes include taking measurements using the measurement and testing device 100 of FIG. 1 and recording the measurement and testing device data 522 results in the measurement and testing database 220. A portion of the processes are performed in the server 120, a plurality of processors 128, communication devices 226, artificial intelligence 122 device with integrated machine learning 124 functions is implemented using at least one set of computer-readable instructions 126, including communicating the results to clinicians and specific patients.

The cancer companion diagnostic direct apoptosis assay of purified cancer cells includes reporting test results, interpretations, and the suggested clinician decision tree with a digital application to a clinician's digital device 340 of FIG. 3 for allowing a clinician and patient to discuss a course of treatment based on the results of the testing for that specific patient, in one embodiment.

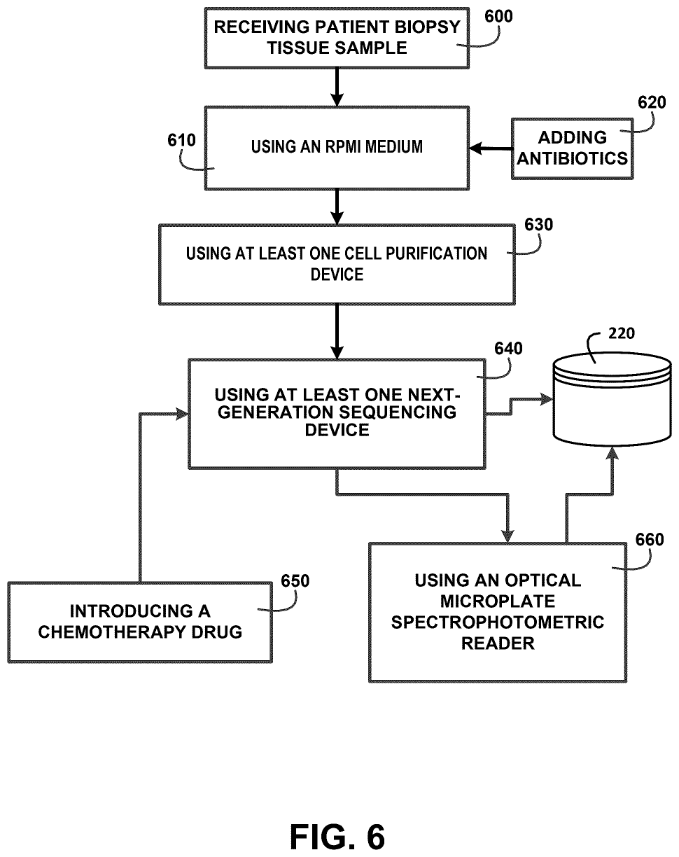

FIG. 6 shows a block diagram of an overview flow chart of receiving patient biopsy tissue samples in one embodiment. FIG. 6 shows processes for the cancer companion diagnostic direct apoptosis assay of purified cancer cells 500 of FIG. 5 that includes receiving a patient biopsy tissue sample 600. The method consists of using an RPMI medium 610 or other medium with or without other additives to preserve a cancer biopsy. The process consists of adding antibiotics 620 to a portion of the preserved cancer biopsy. Preparation of the cancer cells for testing includes using at least one cell purification device 630 including as a magnetic cell separation or flow cytometric cell sorter to purify and sort out cancer cells. Individual tests on the cancer cells are performed using at least one next-generation sequencing device 640 to perform an analysis using an optical microplate spectrophotometric reader 660 in addition to direct apoptosis testing. The testing data and results are recorded in the measurement and testing database 220.

Direct apoptosis testing includes introducing a chemotherapy drug 650 alone, or in combination with other medicines, including cannabinoids/CBD or immunotherapy, to the purified cancer cells. The apoptosis effect of the chemotherapy drug alone, or in combination with other drugs, including cannabinoids/CBD or immunotherapy, on the purified cancer cells is determined using an optical microplate spectrophotometric reader 110 of FIG. 1 to measure the level of apoptosis in cancer cells of one embodiment.

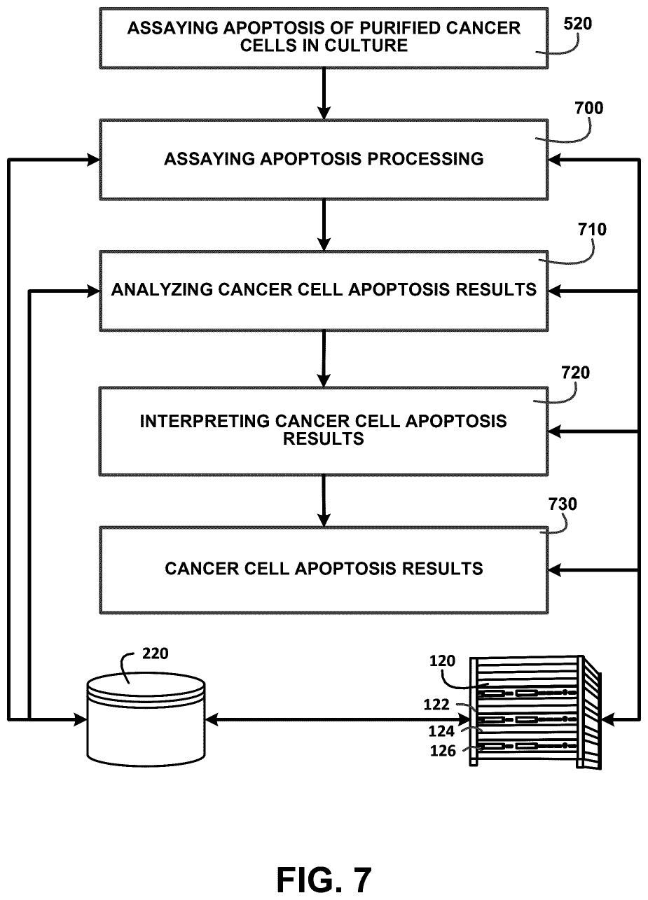

FIG. 7 shows a block diagram of an overview flow chart of assaying apoptosis of purified cancer cells in the culture of one embodiment. FIG. 7 shows processing, including assaying apoptosis of purified cancer cells in culture 520. The process includes patient genomic testing using cells from the preserved cancer biopsy and may consist of analysis of patient blood samples. The effectiveness of various treatments may vary depending on the patient's genetic makeup. The assaying apoptosis processing 700 may include analyzing patient genomic testing for detecting genetic markers associated with cancer, drug resistance, or allergy. This analysis also allows for the assessment of where DNA mutations exist, for example, in a tumor or due to bloodline mutations. The genomic testing data is stored in the measurement and testing database 220.

Analyzing cancer cell apoptosis results 710 from a single chemotherapy drug alone, or in combination with other drugs, including cannabinoids/CBD or immunotherapy, identifies the potential success of a treatment for the single chemotherapy drug alone, or in combination with other drugs, including cannabinoids/CBD or immunotherapy. The cancer cell apoptosis analysis results data are stored in the measurement and testing database 220. Interpreting cancer cell apoptosis results 720 from a single chemotherapy drug alone, or in combination with other drugs, including cannabinoids/CBD or immunotherapy, assists a clinician in evaluating the testing results. Correlating analyses of genetic markers detection, cancer cell apoptosis results 730, and interpretations of the cancer cell apoptosis results are used in the processes. The data stored in the measurement and testing database 220 is used for processing on the server 120, and the results of the processing are also stored in the measurement and testing database 220 of one embodiment. The artificial intelligence 122 device with integrated machine learning 124 functions is implemented using at least one set of computer-readable instructions 126.

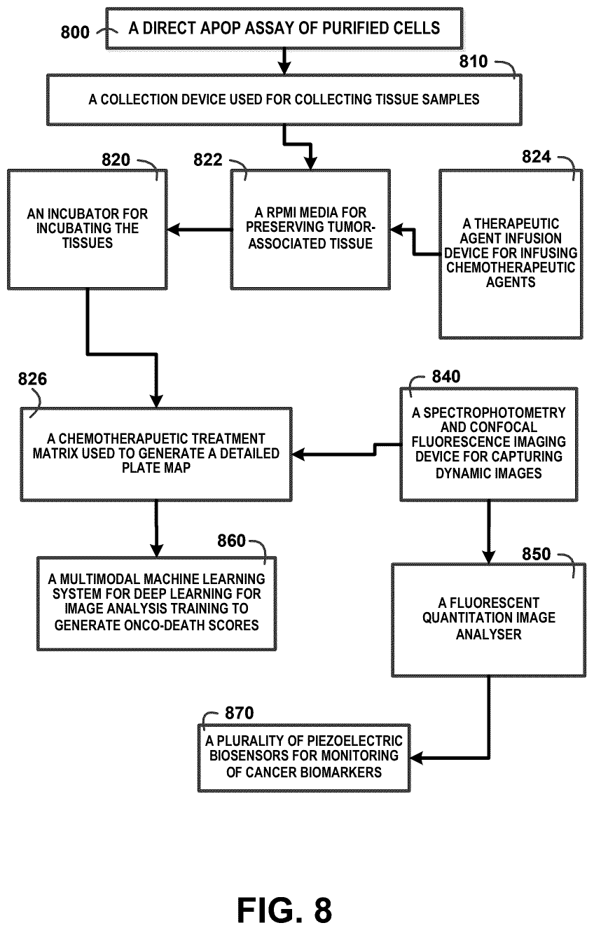

FIG. 8 shows a block diagram of an overview of a method for a direct APOP assay of purified cells of one embodiment. FIG. 8 shows performing a direct APOP assay of purified cells 800. A collection device used for collecting tissue samples 810 in an operating room. An incubator for incubating the tissues 820 collected in suspension in the presence of therapeutic agents. An RPMI media for preserving tumor-associated tissue 822 and shipping the tissues to a laboratory. A therapeutic agent infusion device for infusing chemotherapeutic agents 824 into the microplate wells holding tumor-associated tissue samples. A chemotherapeutic treatment matrix used to generate a detailed plate map 826, of which microplate wells have been exposed to chemotherapeutic agents. A spectrophotometry and confocal fluorescence imaging device for capturing dynamic images 840 of the incubated cancer cells. A multimodal machine learning 124 of FIG. 1 system for deep learning for image analysis training to generate oncological-death scores 860. A fluorescent quantitation image analyzer 850 wherein the images are gated and analyzed for signs and evidence of apoptosis. A plurality of piezoelectric biosensors for monitoring of cancer biomarkers 870.

A method for direct APOP assay of purified cells, including performing a direct APOP assay of purified cells. The method for direct APOP assay of purified cells includes performing the assays on patient-purified cells to assess the effectiveness of drug treatments specific to that patient's current condition, including genetics and prior treatment effects. Performing a direct APOP assay of purified cells 800 includes assaying apoptosis of purified cells passaged in culture and determining antitumor activity or other effects by growth inhibition or other methods and using APOP for anti-inflammatory therapy (e.g., for inflammatory disease, sarcoidosis, granulomatosis diseases, arthritis, colitis, inflammatory skin diseases, myocardial diseases, lung diseases, neurological diseases, liver diseases) and using APOP for anti-immunological therapy (e.g., for autoimmune diseases, multiple sclerosis, transplant rejection) and using APOP to increase immune therapy effects (e.g., for cancer, leukemia, or other neoplastic disease). Using the APOP assay on the therapy of patients with resistant or heavily pretreated cancer, and the clinician and/or the patient are considering no further standard chemotherapy.

The method for direct APOP assay of purified cells includes interpreting APOP results for a series of drugs or combinations for suggested clinician decisions in choosing potential treatments. The clinicians may receive the direct APOP assay and suggested clinician decisions using a direct APOP assay of purified cells application installed on a clinician's mobile device 130 of FIG. 1 with an application 132 of FIG. 1 including a smartphone, digital tablet, or computer. The method for direct APOP assay of purified cells is used for enhancing drug development decisions by use of APOP assay and cell growth inhibition, identifying non-equivalences of drugs, identifying an anti-apoptosis drug, evaluating whether to consider using immunoactive drugs to treat cancer, promoting immune therapy effects of immuno-active medications and/or immune cells in treating cancer or leukemia, and reducing the cost of chemotherapy and/or drug therapy for cancer in one embodiment.

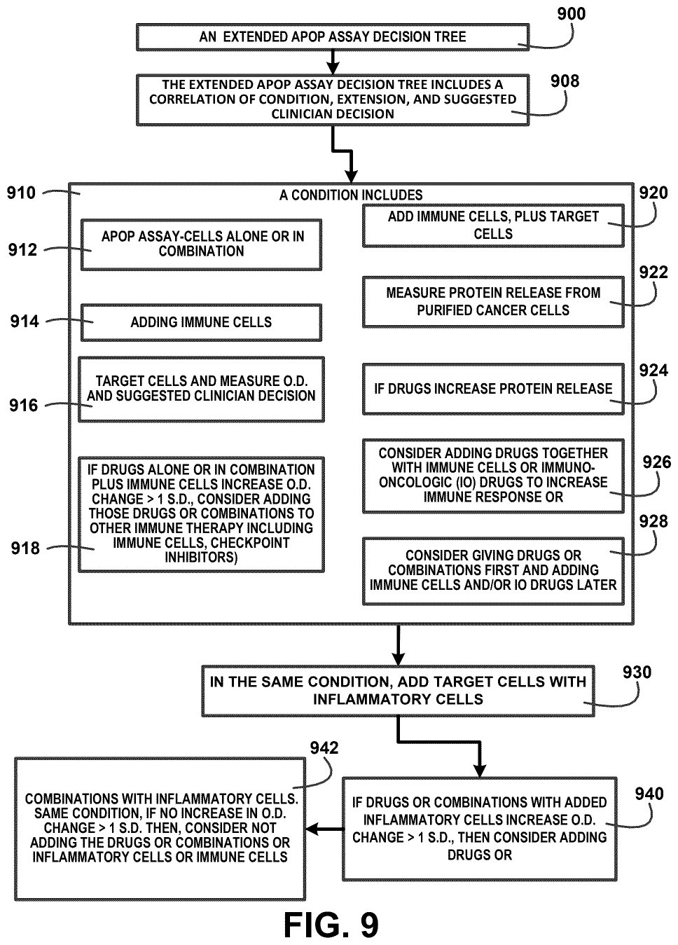

FIG. 9 shows a block diagram of an overview of the extended APOP assay decision tree of one embodiment. FIG. 9 shows the extended APOP assay decision tree 900 of one embodiment. The block diagram shows the extended APOP assay decision tree. The extended APOP assay decision tree includes a correlation of condition, extension, and suggested clinician decision 908. A condition includes 910, for example, APOP assay-cells alone or in combination 912, an extension, for example, adding immune cells 914 (car-t cells or modified lymphocytes), target cells and measure O.D. and suggested clinician decision 916, for example, if drugs alone or in combination plus immune cells increase O.D. change>1 S.D., consider adding those drugs or combinations to other immune therapy (e.g. immune cells, checkpoint inhibitors) 918.

The extended APOP assay decision tree continues with the same, add immune cells, plus target cells 920 and measure protein release from purified cancer cells 922, if drugs increase protein release 924; and consider adding drugs together with immune cells or immuno-oncologic (IO) drugs to INCREASE immune response 926 or consider giving drugs or combinations first and adding immune cells and/or IO drugs later 928. In the same condition, add target cells with inflammatory cells 930. If drugs or combinations with added inflammatory cells increase O.D. change>1 S.D., then consider adding drugs or 940 combinations with inflammatory cells. If there is no increase in O.D. change>1 S.D., then consider not adding the drugs, combinations, or inflammatory cells or immune cells, 942 of one embodiment.

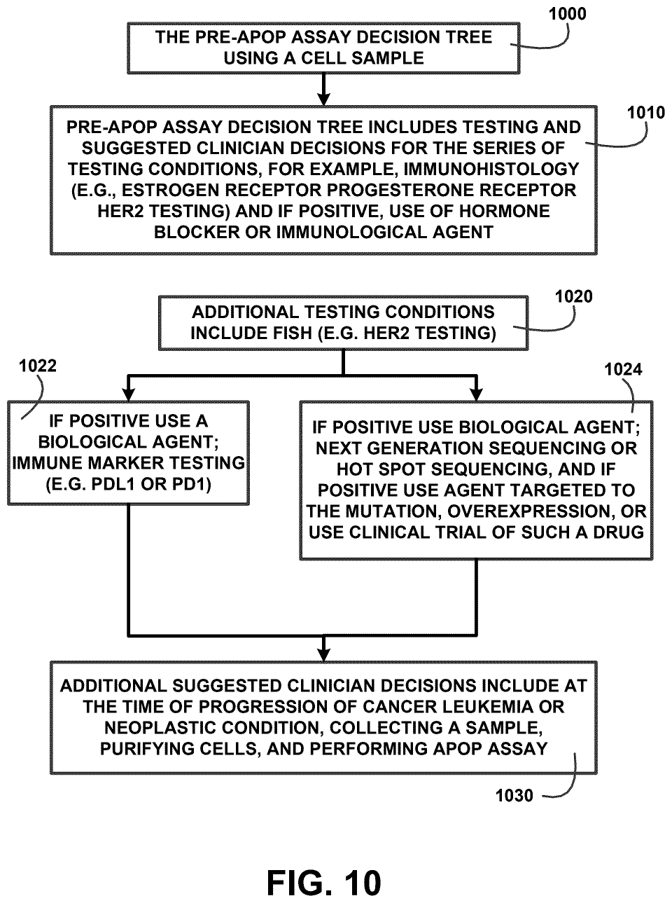

FIG. 10 shows a block diagram of an overview of the pre-APOP assay decision tree of one embodiment. FIG. 10 shows the pre-APOP assay decision tree of one embodiment—the pre-APOP assay decision tree using a cell sample 1000. The pre-APOP assay decision tree includes testing and suggested clinician decisions for the series of testing conditions, for example, immunohistology (e.g., estrogen receptor, progesterone receptor, HER2 testing), and if positive, use of a hormone blocker or immunological agent 1010.

Additional testing conditions include fish (e.g. HER2 testing) 1020, if positive use a biological agent; immune marker testing (e.g. PDL1 or PD1) 1022, and if positive use an immunological agent; flow cytometry (to measure targets or markers), and if positive use biological agent; next generation sequencing or hot spot sequencing, and if positive use agent targeted to the mutation, overexpression, or use clinical trial of such a drug 1024. Additional suggested clinician decisions include, at the time of progression of cancer, leukemia, or a neoplastic condition, collecting a sample, purifying cells, and performing the APOP assay 1030 of one embodiment.

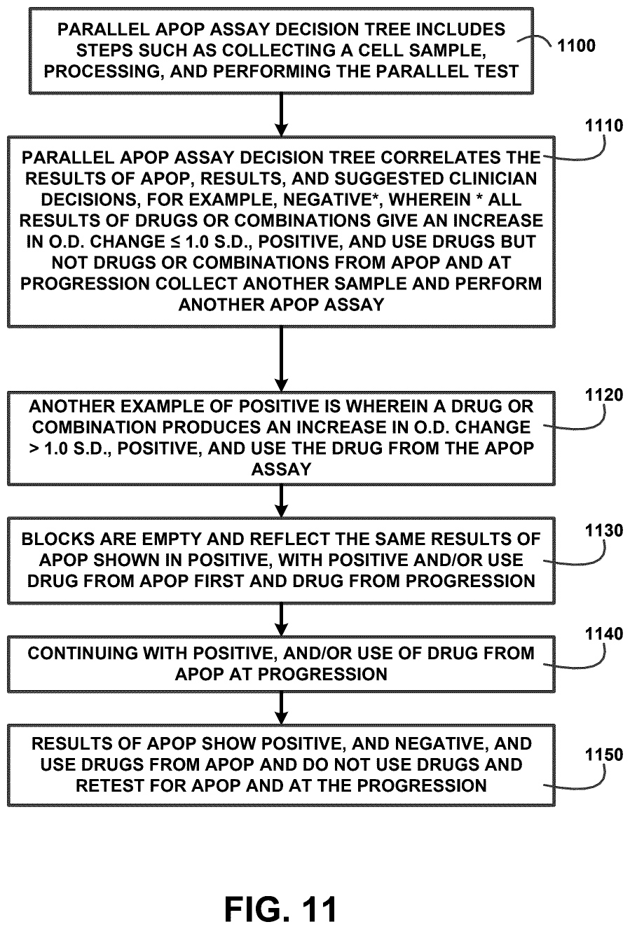

FIG. 11 shows a block diagram of an overview of the parallel APOP assay decision tree of one embodiment. FIG. 11 shows the parallel APOP assay decision tree of one embodiment. The parallel APOP assay decision tree includes steps including collecting a cell sample, processing, and performing the parallel test 1100. The parallel APOP assay decision tree correlates the results of APOP, results, and suggested clinician decisions, for example, negative*, wherein * all results of drugs or combinations give an increase in O.D. change≤1.0 S.D., positive, and use drugs but not drugs or combinations from APOP and at progression collect another sample and perform another APOP assay 1110. Another example of positive is wherein a drug or combination produces an increase in O.D. change>1.0 S.D., positive, and use the drug from the APOP assay 1120.

Blocks are empty and reflect the same results of APOP shown in positive, with positive and/or use drug from APOP first and drug from progression 1130. Continuing with positive, and/or use of drug from APOP at progression 1140. Results of APOP show positive, and negative, and use drugs from APOP and do not use drugs, and retest for APOP, and at the progression 1150 of one embodiment.

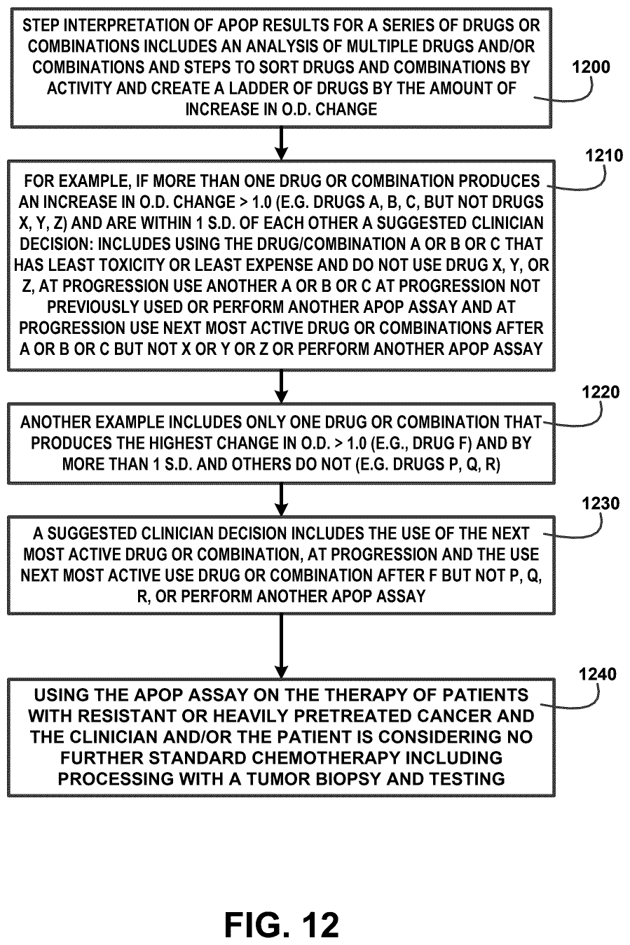

FIG. 12 shows a block diagram of an overview of the interpretation of APOP results for a series of drugs or combinations of one embodiment. FIG. 12 shows the interpretation of APOP results for a series of drugs or combinations of one embodiment. The block diagram shows a step-by-step interpretation of APOP results for a series of drugs or combinations. Step interpretation of APOP results for a series of drugs or combinations, including an analysis of multiple drugs and/or combinations, and steps to sort drugs and combinations by activity and create a ladder of drugs by the amount of increase in O.D. change 1200. For example, if more than one drug or combination produces an increase in O.D. change>1.0 (e.g. drugs A, B, C, but not drugs X, Y, Z) and are within 1 S.D. of each other a suggested clinician decision: includes using the drug/combination A or B or C that has least toxicity or least expense and do not use drug X, Y, or Z, at progression use another A or B or C at progression not previously used or perform another APOP assay and at progression use next most active drug or combinations after A or B or C but not X or Y or Z or perform another APOP assay 1210 of one embodiment.

Another example includes only one drug or combination that produces the highest change in O.D.>1.0 (e.g., drug F) and by more than 1 S.D., and others do not (e.g., drugs P, Q, R) 1220. A suggested clinician decision includes the use of the next most active drug or combination at progression, and the use of the next most active drug or combination after F, but not P, Q, R, or perform another APOP assay 1230 of one embodiment.

The following block diagram shows an overview of using the APOP assay in the therapy of patients with resistant or heavily pretreated cancer in one embodiment. The APOP assay is used to treat patients with resistant or heavily pretreated cancer, and the clinician and/or the patient are considering no further standard chemotherapy. Using the APOP assay on the therapy of patients with resistant or heavily pretreated cancer, and the clinician and/or the patient are considering no further standard chemotherapy, including processing with a tumor biopsy, and testing 1240.

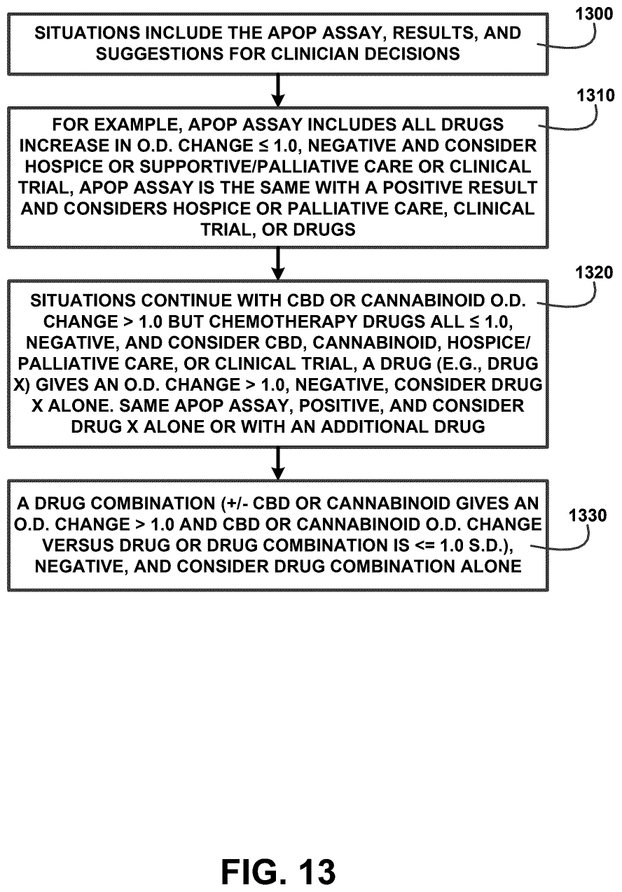

FIG. 13 shows a block diagram of an overview of situations of one embodiment. FIG. 13 shows situations including the APOP assay, results, and suggestions for clinician decisions 1300. For example, the APOP assay includes all drugs with an increase in O.D. change≤1.0, negative, and considers hospice or supportive/palliative care or clinical trial. APOP assay is the same as a positive result and considers hospice or palliative care, clinical trial, or drugs 1310. The situations continue with CBD or cannabinoid O.D. change>1.0 but chemotherapy drugs all≤1.0, negative, and consider CBD, cannabinoid, hospice/palliative care, or clinical trial. A drug (e.g., drug X) gives an O.D. change>1.0, negative, consider drug X alone. Same APOP assay, positive, and consider drug X alone or with an additional drug 1320. A drug combination (+/−CBD or cannabinoid gives an O.D. change>1.0 and CBD or cannabinoid O.D. change versus drug or drug combination is <=1.0 S.D.), negative, and consider drug combination alone 1330 of one embodiment.

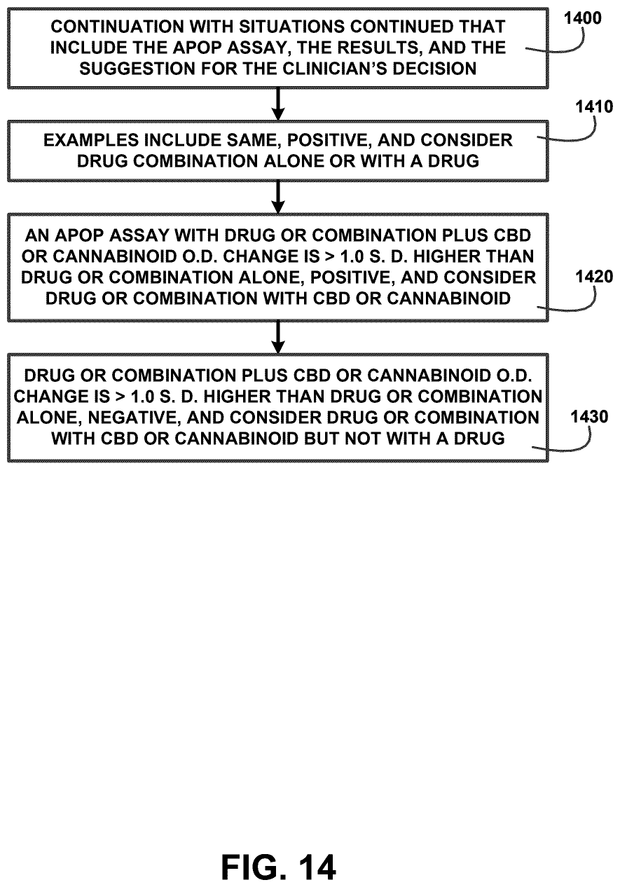

FIG. 14 shows a block diagram of an overview of situations continued in one embodiment. FIG. 14 shows a continuation of situations that include the APOP assay, the results, and the suggestion for the clinician's decision 1400. Examples include the same, positive, and consider drug combination alone or with a drug 1410. An APOP assay with drug or combination plus CBD or cannabinoid O.D. change is>1.0 S. D. higher than drug or combination alone, positive, and considered drug or combination with CBD or cannabinoid 1420. Drug or combination plus CBD or cannabinoid O.D. change is>1.0 S. D. higher than drug or combination alone, negative, and consider drug or combination with CBD or cannabinoid but not with a drug 1430 of one embodiment.

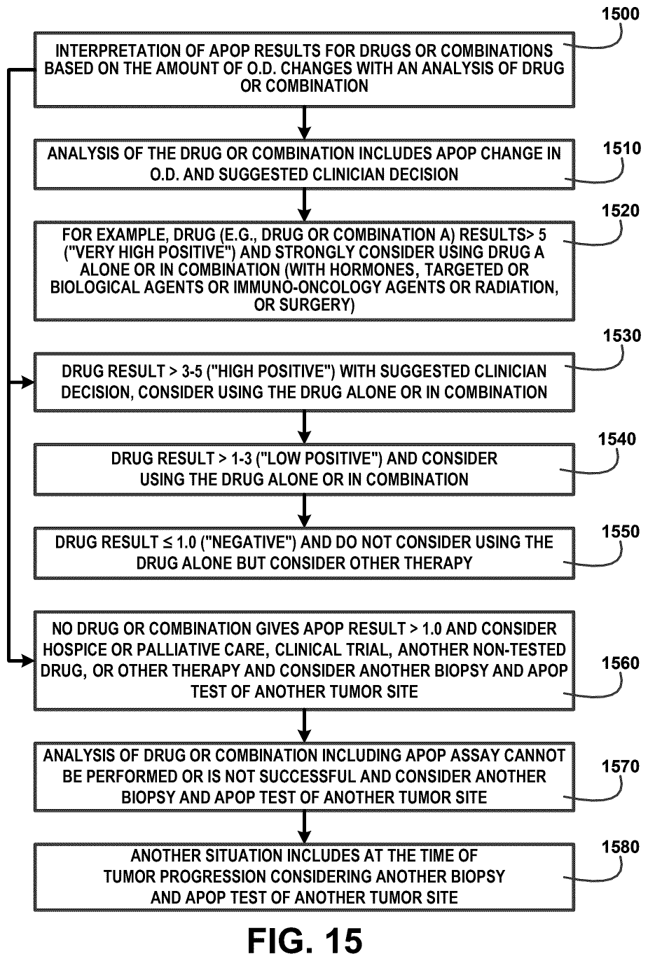

FIG. 15 shows a block diagram of an overview of the interpretation of APOP results for drugs or combinations based on the amount of O.D. change in one embodiment. FIG. 15 shows the interpretation of APOP results for drugs or combinations based on the amount of O.D. change of one embodiment. Interpretation of APOP results for drugs or combinations based on the amount of O.D. changes with an analysis of drug or combination 1500. The analysis of the drug or combination includes APOP change in O.D. and suggested clinician decision 1510. For example, drug (e.g., drug or combination A) results>5 (“very high positive”) and strongly consider using drug A alone or in combination (with hormones, targeted or biological agents, immuno-oncology agents, or radiation, or surgery) 1500.

Drug result>3-5 (“high positive”) with suggested clinician decision, consider using the drug alone or in combination 1530. Drug result>1-3 (“low positive”) and consider using the drug alone or in combination 1240. Drug result≤1.0 (“negative”) and do not consider using the drug alone, but consider other therapy 1550. No drug or combination gives an APOP result>1.0, and consider hospice or palliative care, clinical trial, another non-tested drug, or other therapy, and consider another biopsy and an APOP test of another tumor site 1260. If the analysis of the drug or combination, including the APOP assay, cannot be performed or is not successful, consider another biopsy and APOP test of another tumor site 1570. Another situation includes, at the time of tumor progression, considering another biopsy and APOP test of another tumor site 1580 of one embodiment.

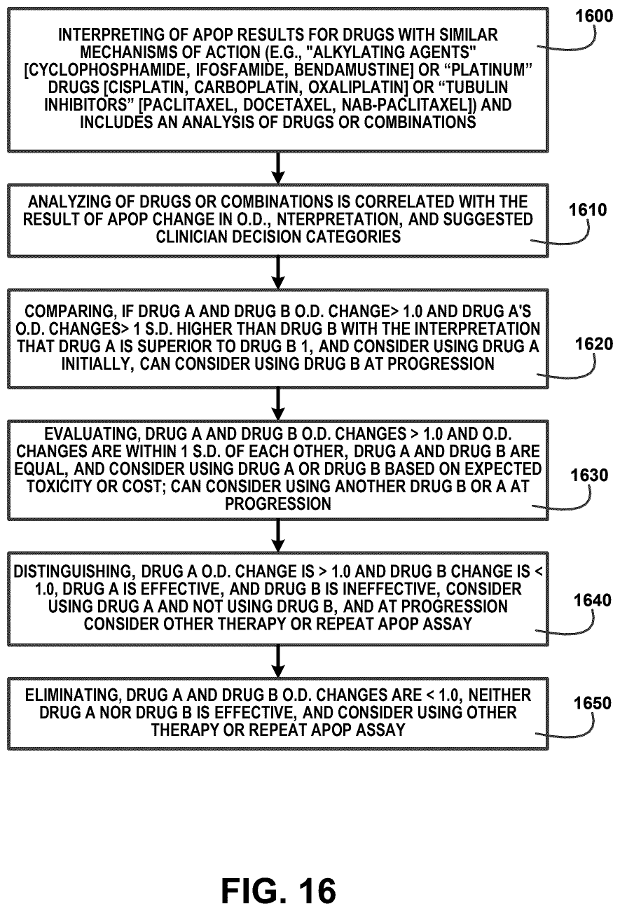

FIG. 16 shows a block diagram of an overview of the interpretation of APOP results for drugs with similar mechanisms of action of one embodiment. FIG. 16 shows the interpretation of APOP results for drugs with similar mechanisms of action of one embodiment. The interpretation of APOP results for drugs with similar mechanisms of action (e.g., “alkylating agents” [cyclophosphamide, ifosfamide, bendamustine] or “platinum” drugs [cisplatin, carboplatin, oxaliplatin] or “tubulin inhibitors” [paclitaxel, docetaxel, nab-paclitaxel]) and includes an analysis of drugs or combinations 1600. The analysis of drugs or combinations is correlated with the result of APOP change in O.D., interpretation, and suggested clinician decision categories 1610. For example, if drug A and drug B O.D. change>1.0 and drug A′s O.D. changes>1 S.D. higher than drug B, with the interpretation that drug A is superior to drug B 1, and considering using drug A initially, one can consider using drug B at progression 1620.

Drug A and drug B O.D. changes>1.0 and O.D. changes are within 1 S.D. of each other, drug A and drug B are equal, and consider using drug A or drug B based on expected toxicity or cost; can consider using another drug B or A at progression 1630. Drug A O.D. change is>1.0, and drug B change is<1.0. Drug A is effective, and drug B is ineffective. Consider using drug A and not using drug B, and at progression, consider other therapy or repeat the APOP assay 1640. Drug A and drug B O.D. changes are<1.0, neither drug A nor drug B is effective, and consider using other therapy or repeat the APOP assay 1650 of one embodiment.

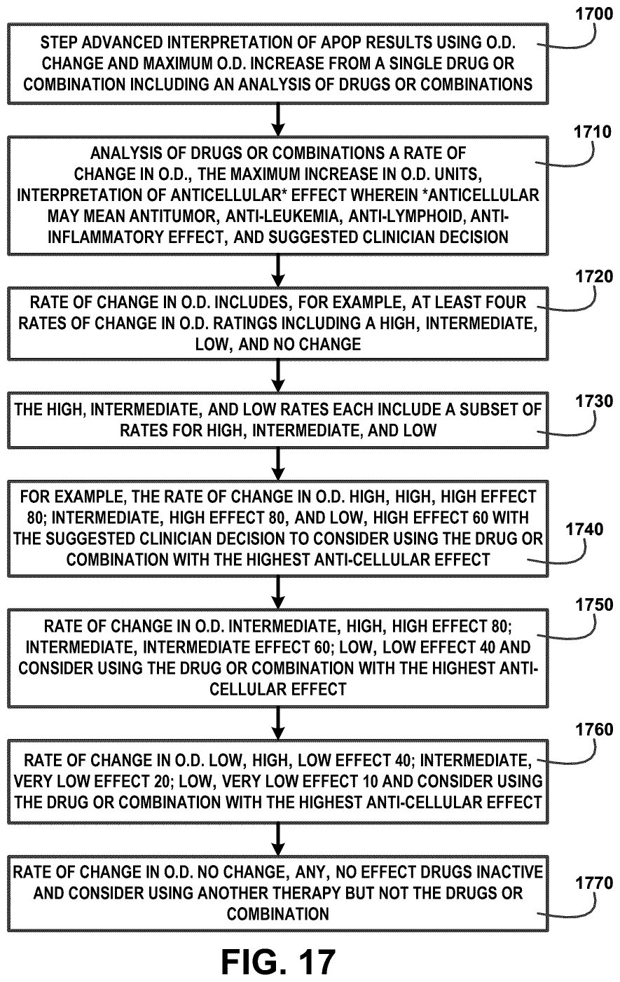

FIG. 17 shows a block diagram and an overview of the advanced interpretation of APOP results using O.D. change and maximum O.D. increase from a single drug or a combination of one embodiment. FIG. 17 shows the advanced interpretation of APOP results using O.D. change and maximum O.D. increase from a single drug or combination. The step advanced interpretation of APOP results using O.D. change and maximum O.D. increase from a single drug or combination, including an analysis of drugs or combinations 1700. The analysis of drugs or combinations of a rate of change in O.D., the maximum increase in O.D. units, interpretation of anticellular* effect, wherein *anticellular may mean antitumor, anti-leukemia, anti-lymphoid, anti-inflammatory effect, and suggested clinician decision 1710 of one embodiment.

The rate of change in O.D. includes, for example, at least four rates of change in O.D. ratings, including a high, intermediate, low, and no change 1720. The high, intermediate, and low rates each include a subset of rates for high, intermediate, and low 1730. For example, the rate of change in O.D. high, high, high effect 80; intermediate, high effect 80, and low, high effect 60, with the suggested clinician decision to consider using the drug or combination with the highest anti-cellular effect 1740 of one embodiment.

Rate of change in O.D. intermediate, high, high effect 80; intermediate, intermediate effect 60; low, low effect 40, and consider using the drug or combination with the highest anti-cellular effect 1750 of one embodiment.

Rate of change in O.D. low, high, low effect 40; intermediate, very low effect 20; low, very low effect 10, and consider using the drug or combination with the highest anti-cellular effect 1760. Rate of change in O.D.: no change, any, no effect, drugs inactive, and consider using another therapy, but not the drugs or combination 1770 of one embodiment.

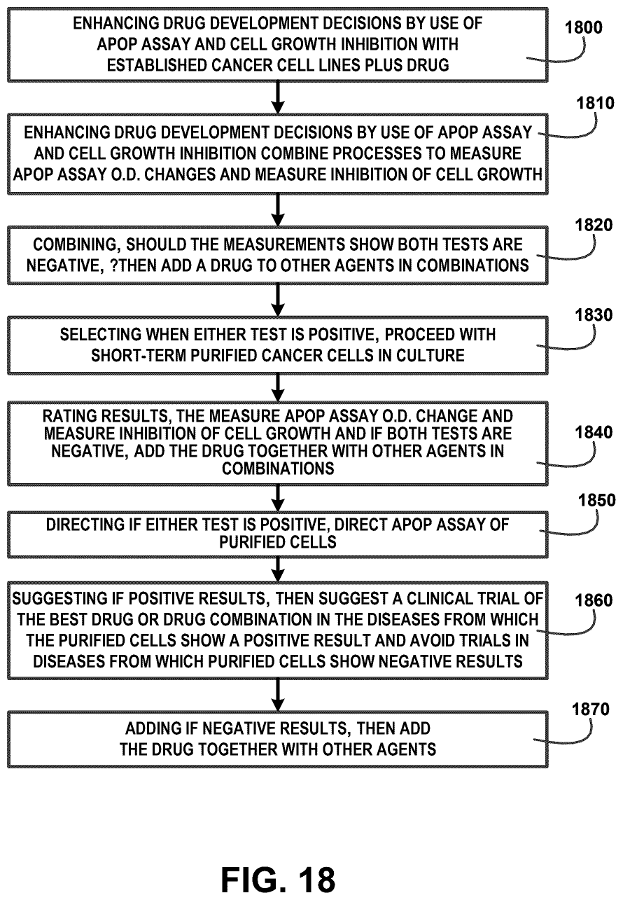

FIG. 18 shows a block diagram of an overview of enhancing drug development decisions by use of the APOP assay and cell growth inhibition of one embodiment. FIG. 18 shows enhancing drug development decisions by the use of the APOP assay and cell growth inhibition of one embodiment. Enhancing drug development decisions by use of the APOP assay and cell growth inhibition with established cancer cell lines plus drug 1800. The enhancement of drug development decisions by use of the APOP assay and cell growth inhibition combines processes to measure APOP assay O.D. changes and measure inhibition of cell growth 1810. Should the measurements show both tests are negative, then add a drug to other agents in combinations 1820 of one embodiment.

When either test is positive, proceed with short-term purified cancer cells in culture 1830. Measure the APOP assay O.D. change and measure inhibition of cell growth, and if both tests are negative, add the drug together with other agents in combination 1840. If either test is positive, direct APOP assay of purified cells 1850. If positive results, then suggest a clinical trial of the best drug or drug combination in the diseases from which the purified cells show a positive result, and avoid trials in diseases from which purified cells show negative results 1860. If negative results, then add the drug together with other agents, 1870 of one embodiment.

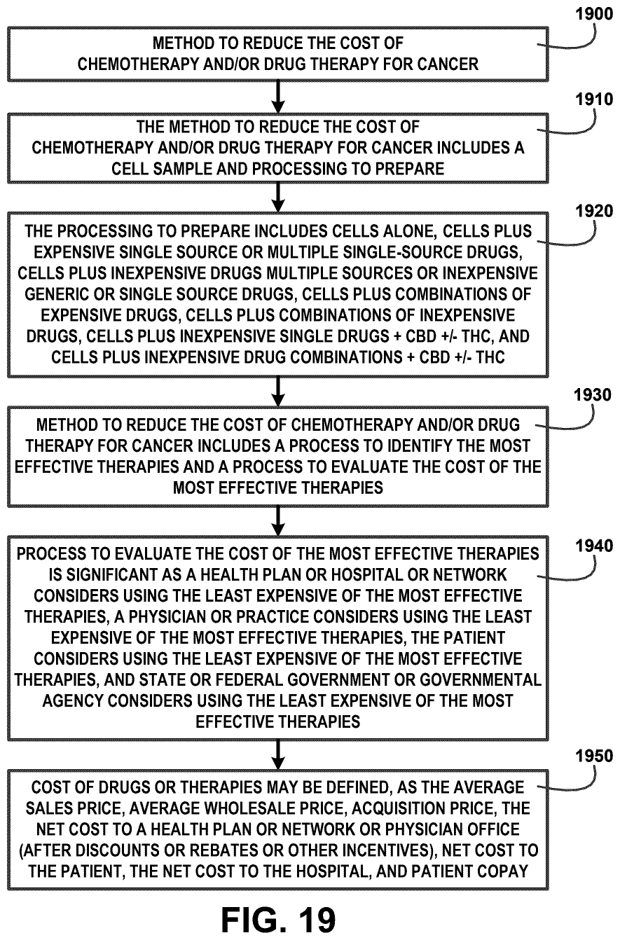

FIG. 19 shows a block diagram of an overview of a method to reduce the cost of chemotherapy and/or drug therapy for cancer of one embodiment. FIG. 19 shows a method to reduce the cost of chemotherapy and/or drug therapy for cancer 1900. The method to reduce the cost of chemotherapy and/or drug therapy for cancer includes a cell sample and processing to prepare 1910. The processing to prepare includes cells alone, cells plus expensive single source or multiple single-source drugs, cells plus inexpensive drugs multiple sources or inexpensive generic or single source drugs, cells plus combinations of expensive drugs, cells plus combinations of inexpensive drugs, cells plus inexpensive single drugs+CBD +/−THC, and cells plus inexpensive drug combinations+CBD +/−THC 1920.

The method to reduce the cost of chemotherapy and/or drug therapy for cancer includes a process to identify the most effective therapies and a process to evaluate the cost of the most effective therapies 1930.

The process to evaluate the cost of the most effective therapies is significant as a health plan or hospital or network considers using the least expensive of the most effective therapies, a physician or practice considers using the least expensive of the most effective therapies 1940, the patient considers using the least expensive of the most effective therapies, and state or federal government or governmental agency considers using the least expensive of the most effective therapies of one embodiment.

The ensuing block diagram shows an overview of the cost of drugs or therapies, defined of one embodiment. The cost of drugs or therapies may be defined, as the average sales price, average wholesale price, acquisition price, the net cost to a health plan or network or physician office (after discounts or rebates or other incentives), net cost to the patient, the net cost to the hospital, and patient copay 1950 of one embodiment.

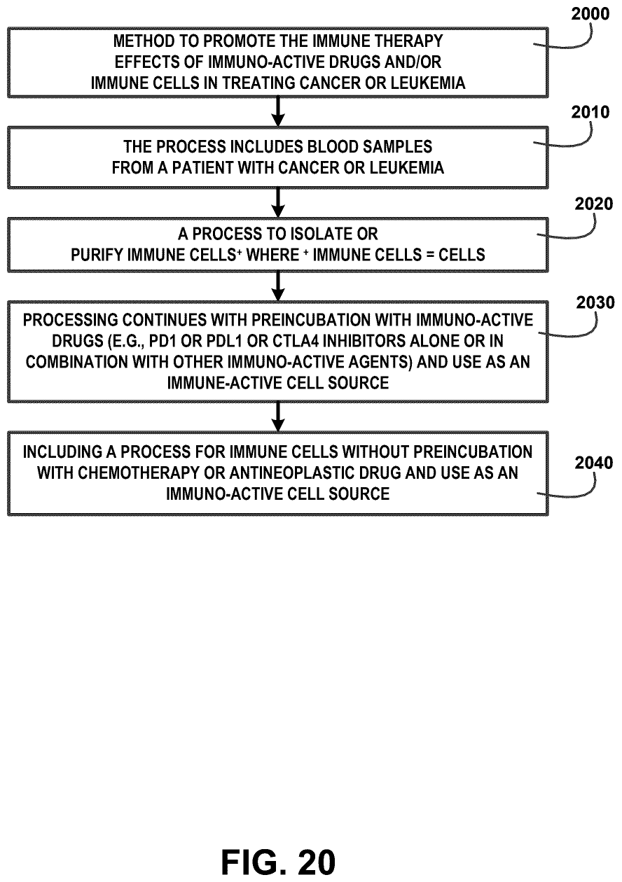

FIG. 20 shows a block diagram of an overview of a method to promote immune therapy effects of immuno-active drugs and/or immune cells in treating cancer or leukemia of one embodiment. FIG. 20 shows a method to promote the immune therapy effects of immuno-active drugs and/or immune cells in treating cancer or leukemia 2000. The process includes blood samples from a patient with cancer or leukemia 2010. A process to isolate or purify immune cells⁺, where ⁺immune cells=cells 2020. Processing continues with preincubation with immuno-active drugs (e.g., PD1 or PDL1or CTLA4 inhibitors alone or in combination with other immuno-active agents) and use as an immuno-active cell source 2030. Including a process for immune cells without preincubation with chemotherapy or antineoplastic drug, and use as an immuno-active cell source 2040 of one embodiment.

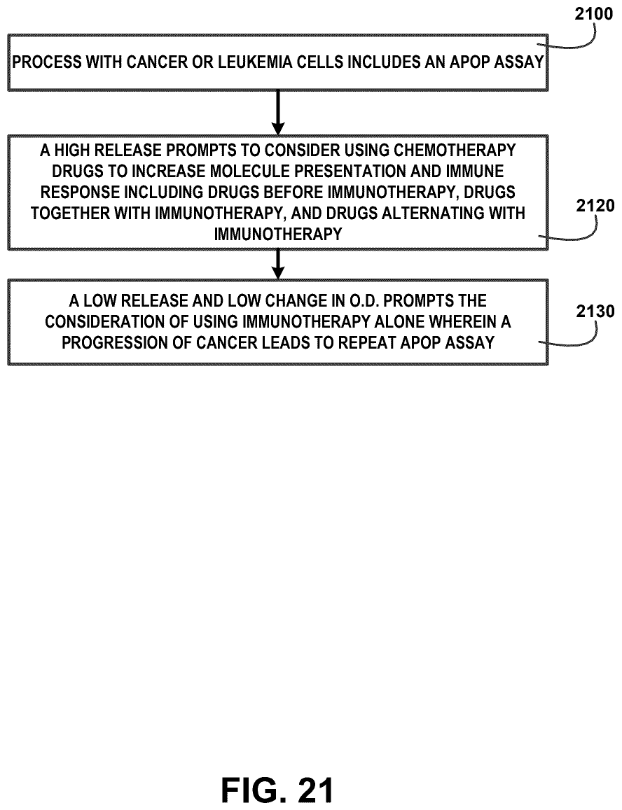

FIG. 21 shows a block diagram of an overview of cancer or leukemia cells of one embodiment. FIG. 21 shows the process for cancer or leukemia cells, which includes an APOP assay 2100. The APOP assay consists of a method to measure molecule release into supernatant culture fluid, where molecule refers to a specific substance, e.g., protein, antigen, or cell component. A high release prompts the consideration of using chemotherapy drugs to increase molecule presentation and immune response, including drugs before immunotherapy, drugs together with immunotherapy, and drugs alternating with immunotherapy. A low release and low change in O.D. prompts the consideration of using immunotherapy alone, wherein a progression of cancer leads to repeat APOP assay 2130 of one embodiment.

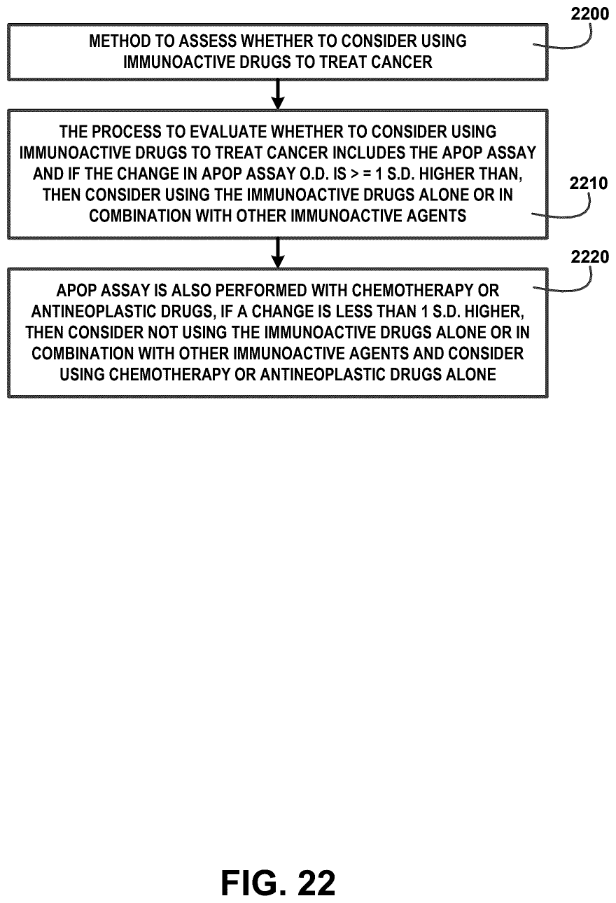

FIG. 22 shows a block diagram of an overview of a method to evaluate whether to consider using immunoactive drugs to treat cancer in one embodiment. FIG. 22 shows a method to assess whether or not to consider using immunoactive drugs to treat cancer 2200. The process to evaluate whether to consider using immunoactive drugs to treat cancer includes the APOP assay, and if the change in APOP assay O.D. is>=1 S.D. higher than, then consider using the immunoactive drugs alone or in combination with other immunoactive agents 2210, in one embodiment.

The APOP assay is also performed with chemotherapy or antineoplastic drugs. If a change is less than 1 S.D. higher, then consider not using the immunoactive drugs alone or in combination with other immunoactive agents, and consider using chemotherapy or antineoplastic drugs alone 2220, in one embodiment.

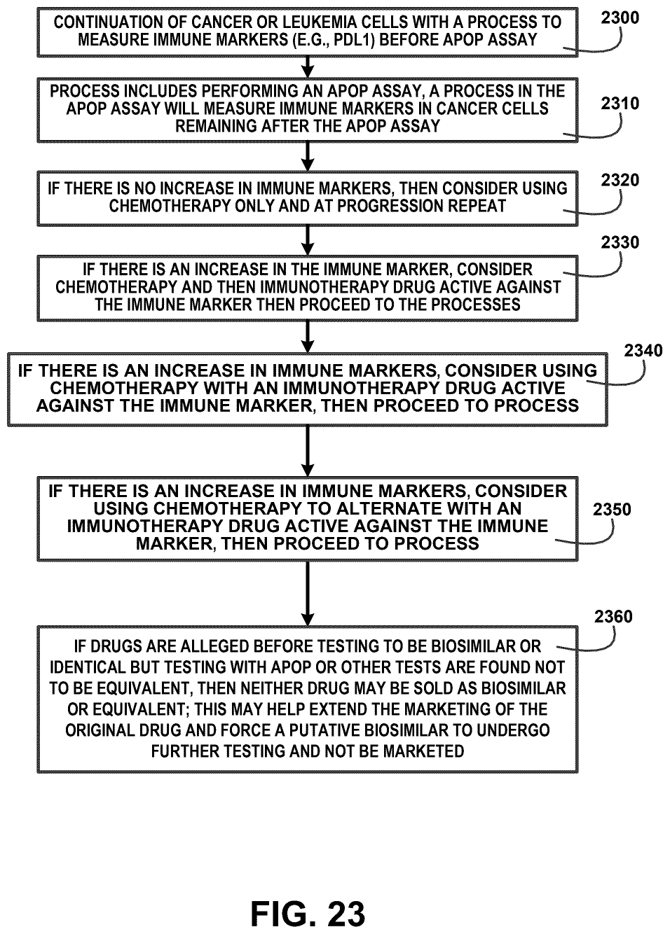

FIG. 23 shows a block diagram of an overview of measuring immune markers before the APOP assay of one embodiment. FIG. 23 shows a continuation of cancer or leukemia cells with a process to measure immune markers (e.g., PDL1) before the APOP assay 2300. The process includes performing an APOP assay. A process in the APOP assay will measure immune markers in cancer cells remaining after the APOP assay 2310. If there is no increase in immune markers, then consider using chemotherapy only, and at progression, repeat 2320. If there is an increase in the immune marker, consider chemotherapy and then an immunotherapy drug active against the immune marker, then proceed to process 2330.

If there is an increase in immune markers, consider using chemotherapy with an immunotherapy drug active against the immune marker, then proceed to process 2340. If there is an increase in immune markers, consider using chemotherapy to alternate with an immunotherapy drug active against the immune marker, then proceed to process 2350. If drugs are alleged before testing to be biosimilar or identical but testing with APOP or other tests are found not to be equivalent, then neither drug may be sold as biosimilar or equivalent; this may help extend the marketing of the original drug and force a putative biosimilar to undergo further testing and not be marketed 2360.

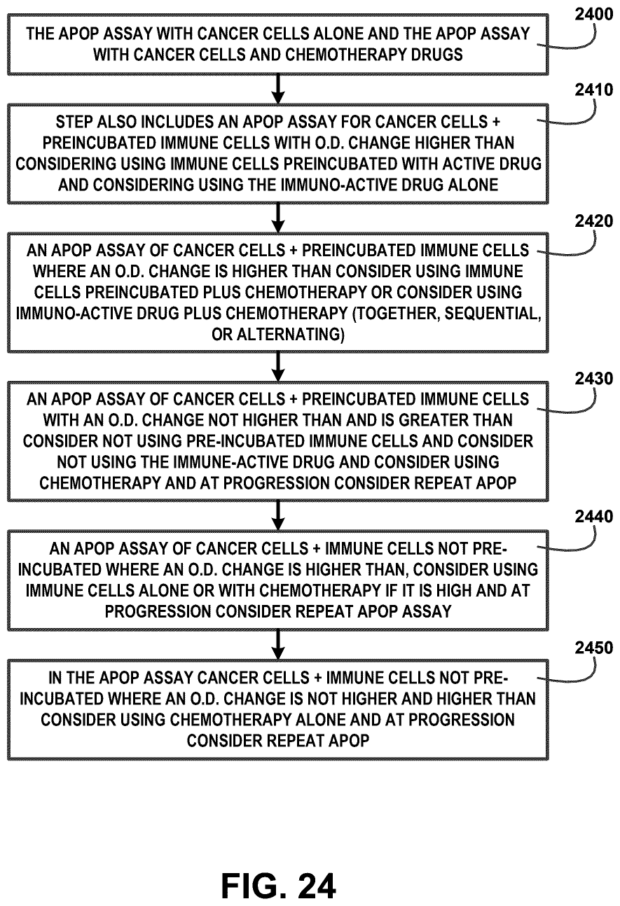

FIG. 24 shows a block diagram of an overview of the APOP assay cancer cells of one embodiment. FIG. 24 shows the step that includes the APOP assay with cancer cells alone and the APOP assay with cancer cells and chemotherapy drugs 2400. The step also includes an APOP assay for cancer cells +preincubated immune cells with O.D. change higher than considering using immune cells preincubated with active drug and considering using the immuno-active drug alone of one embodiment 2410.

An APOP assay of cancer cells+preincubated immune cells, where an O.D. change is higher than considering using immune cells preincubated plus chemotherapy, or consider using immuno-active drug plus chemotherapy (together, sequential, or alternating) of one embodiment 2420. An APOP assay of cancer cells+preincubated immune cells with an O.D. change not higher than and is greater than, consider not using pre-incubated immune cells, consider not using the immune-active drug, consider using chemotherapy, and at progression, consider repeat APOP 2430 of one embodiment. An APOP assay of cancer cells +immune cells not pre-incubated, where an O.D. change is higher than, consider using immune cells alone or with chemotherapy, if it is high, and at progression, consider repeating the APOP assay 2440 of one embodiment. In the APOP assay, cancer cells +immune cells are not pre-incubated, where an O.D. change is not higher, and higher than considered using chemotherapy alone and at progression, consider repeating APOP 2450 of one embodiment.

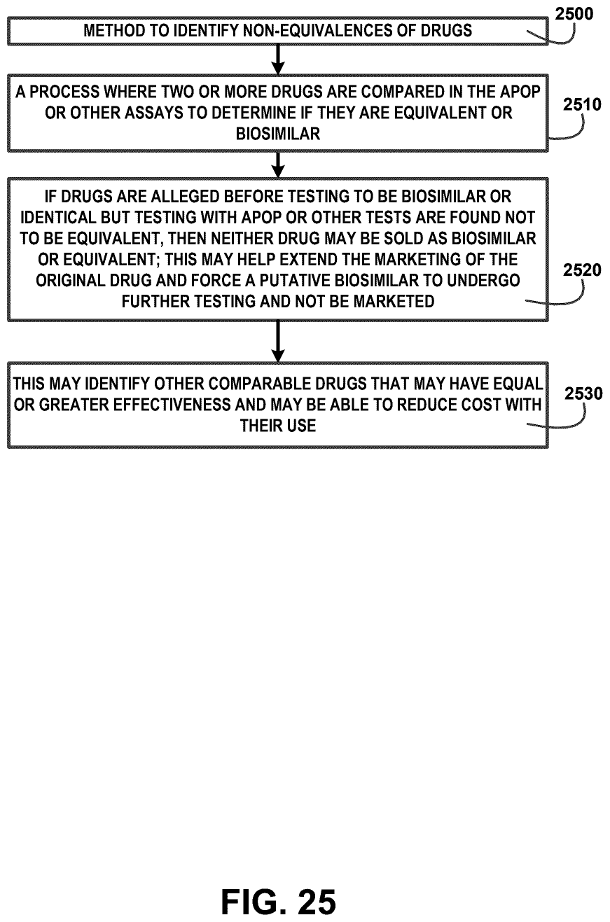

FIG. 25 shows a block diagram of an overview of a method to identify the non-equivalences of drugs of one embodiment. FIG. 25 shows a method to identify the non-equivalences of drugs 2500. A method to identify non-equivalences of drugs 2500 is a process where two or more drugs are compared in the APOP or other assays to determine if they are equivalent or biosimilar 2510. If drugs are alleged before testing to be biosimilar or identical but testing with APOP or other tests are found not to be equivalent, then neither drug may be sold as biosimilar or equivalent; this may help extend the marketing of the original drug and force a putative biosimilar to undergo further testing and not be marketed 2520. This may identify other comparable drugs that may have equal or greater effectiveness and may be able to reduce cost with their use 2530 of one embodiment.

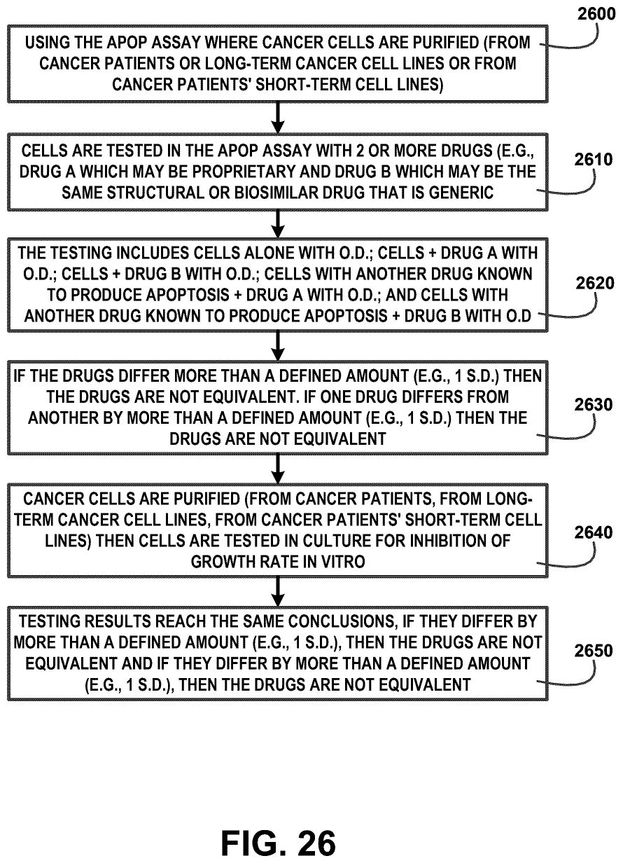

FIG. 26 shows a block diagram of an overview of using the APOP assay of one embodiment. FIG. 26 shows using the APOP assay, where cancer cells are purified (from cancer patients or long-term cancer cell lines or from cancer patients' short-term cell lines) 2600. Cells are tested in the APOP assay with 2 or more drugs (e.g., drug A, which may be proprietary, and drug B, which may be the same structural or biosimilar drug that is generic) of one embodiment 2610.

The testing includes cells alone with O.D.; cells+drug A with O.D.; cells+drug B with O.D.; cells with another drug known to produce Apoptosis+drug A with O.D.; and cells with another drug known to produce Apoptosis+drug B with O.D. 2620. If the drugs differ more than a defined amount (e.g., 1 S.D.), then the drugs are not equivalent. If one drug differs from another by more than a defined amount (e.g., 1 S.D.), then the drugs are not equivalent of one embodiment 2630.

Cancer cells are purified (from cancer patients, from long-term cancer cell lines, from cancer patients' short-term cell lines), then the cells are tested in culture for inhibition of growth rate in vitro 2640. Testing results reach the same conclusions, if they differ by more than a defined amount (e.g., 1 S.D.), then the drugs are not equivalent, and if they differ by more than a defined amount (e.g., 1 S.D.), then the drugs are not equivalent, 2650 of one embodiment.

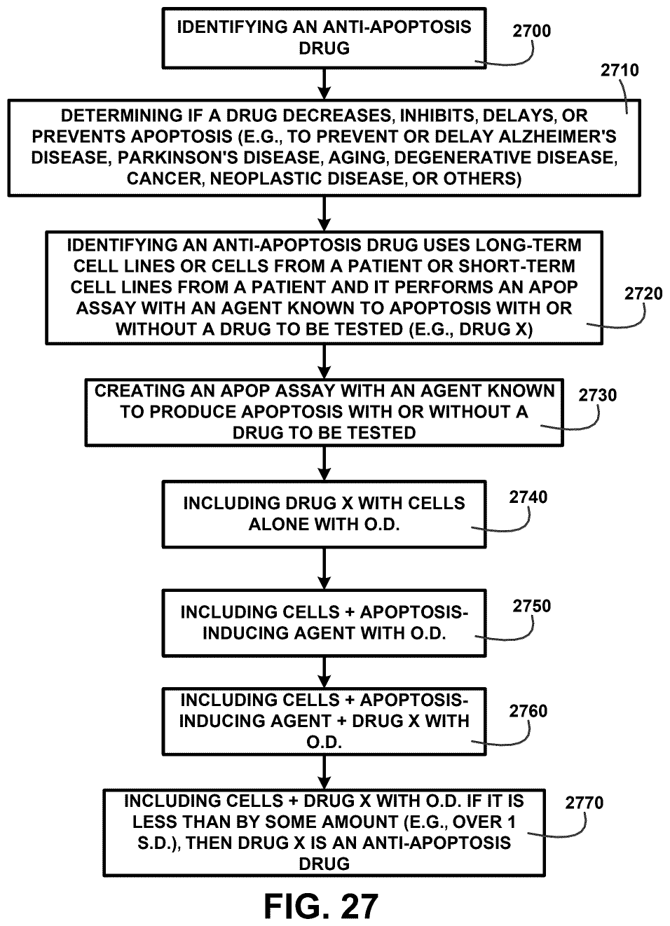

FIG. 27 shows a block diagram of an overview flow chart of a method for identifying an anti-apoptosis drug of one embodiment. FIG. 27 shows a method for identifying an anti-apoptosis drug 2700. This is determining if a drug decreases, inhibits, delays, or prevents Apoptosis (e.g., to prevent or delay Alzheimer's disease, Parkinson's disease, aging, degenerative disease, cancer, Neoplastic disease, or others) 2710.

A method for identifying an anti-apoptosis drug uses long-term cell lines or cells from a patient, or short-term cell lines from a patient. It performs an APOP assay with an agent known to produce Apoptosis with or without a drug to be tested (e.g., drug X) 2720. Creating an APOP assay with an agent known to produce Apoptosis with or without a drug to be tested 2730. Including drug X with cells alone with O.D. 2740. Including cells+Apoptosis-inducing agent with O.D.2750. Including cells+Apoptosis-inducing agent+drug X with O.D. 2760. Including cells+drug X with O.D. If it is less than by some amount (e.g., over 1 S.D.), then drug X is an anti-apoptosis drug 2770.

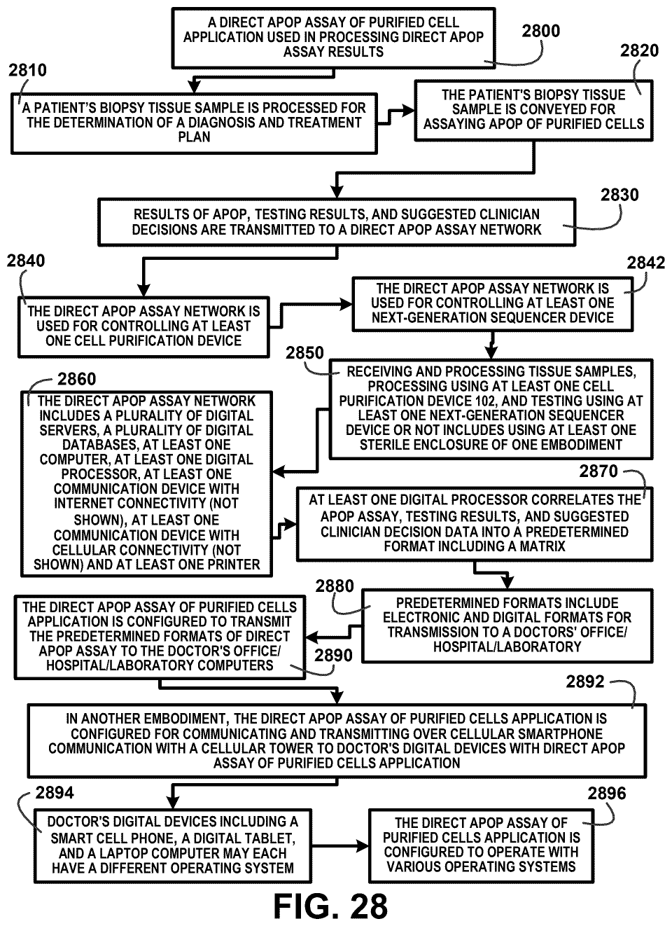

FIG. 28 shows, for illustrative purposes only, an example of a direct APOP assay of purified cells application of one embodiment. FIG. 28 shows a direct APOP assay of purified cell application used in processing direct APOP assay results 2800. A patient visits a doctor's office/hospital/laboratory to provide a tissue sample for a biopsy. A patient's biopsy tissue sample is processed for the determination of a diagnosis and treatment plan 2810. The patient's biopsy tissue sample is conveyed for assaying APOP of purified cells 2820. Results of APOP, testing results, and suggested clinician decisions are transmitted to a direct APOP assay network 2830 to record and perform APOP assay, testing results, and suggested clinician decision correlation matrix.

The direct APOP assay network is used for controlling at least one cell purification device 2840 for purifying tissue sample cells and, for example, long-term cancer cell lines. The direct APOP assay network is used for controlling at least one next-generation sequencer device 2842 used in performing direct APOP assay of purified cell testing. Receiving and processing tissue samples, processing using a cell purification device 104 of FIG. 1, and testing using at least one next-generation sequencer device, or not, includes using at least one sterile enclosure of one embodiment 2850.

The direct APOP assay network includes at least one server 120, a plurality of digital databases, at least one computer, at least one digital processor 128, at least one communication device with internet connectivity (not shown), at least one communication device with cellular connectivity (not shown), and at least one printer 2860. At least one digital processor 128 correlates the APOP assay, testing results, and suggested clinician decision data into a predetermined format, including a matrix 2870. Predetermined formats include electronic and digital formats for transmission to a doctor's office/hospital/laboratory 2880 using different operating systems and computing languages and display formats. In one embodiment, the direct APOP assay of purified cells application is configured to transmit the predetermined formats of direct APOP assay to the doctor's office/hospital/laboratory computers 2890 using internet transmission.

In another embodiment, the direct APOP assay of purified cells application is configured for communicating and transmitting over cellular smartphone communication with a cellular tower to doctors' digital devices with the direct APOP assay of purified cells application 2892. Doctor's mobile device 130 of FIG. 1 with an application 132 of FIG. 1 including a smart cell phone, a digital tablet, and a laptop computer, may each have a different operating system 2894. The direct APOP assay of purified cells application is configured to operate with various operating systems 2896 of one embodiment.

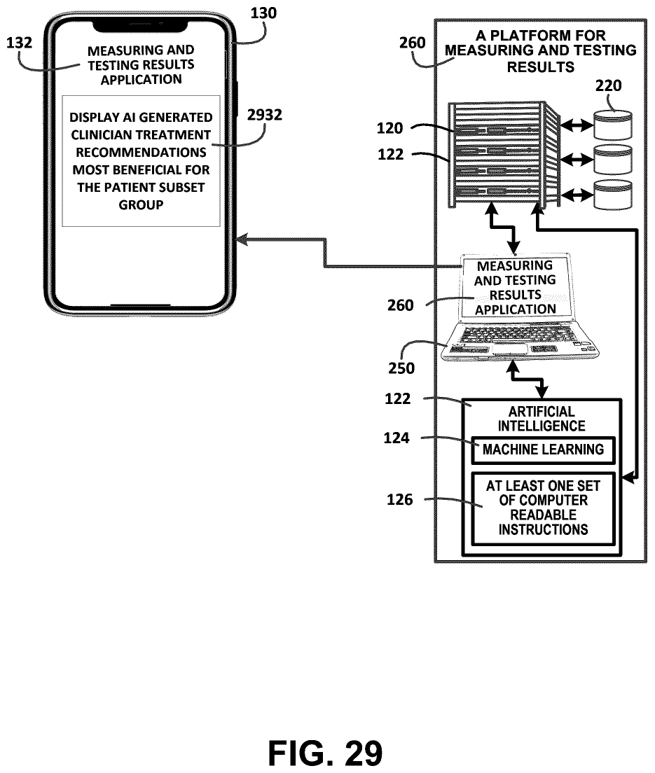

FIG. 29 shows, for illustrative purposes only, an example of a measuring and testing results platform of one embodiment. FIG. 29 shows the platform for measuring and testing results. The platform for measuring and testing results includes recording the results on at least one server 120, at least one artificial intelligence 122 having machine learning 124, a plurality of databases 220, a platform computer 250 having a measuring and testing results application 260, and an artificial intelligence 122 device with integrated machine learning 124 functions. The artificial intelligence 122 device with integrated machine learning 124 functions is implemented using at least one set of computer-readable instructions 126.

A user's mobile device 130, having the measuring and testing results application 132, will receive from the platform computer and display AI-generated clinician treatment recommendations most beneficial for the patient subset group 2932. Using AI in the management of drug therapy because of PGx testing and the ability to use image software to capture and evaluate the thousands of images that will result from the time course experiments, reduces the time taken for evaluations and comparisons of different drug treatment results. Multi-modal AI is used to analyze the images and incorporate the genomic, PGx, and death markers. These AI modules reduce the time to perform the evaluations of the testing outcomes. Classifying tumor subtypes is critical to cancer treatment, as the various subtypes of tumors will respond differently to different types of treatment. AI 122 with machine learning 124 is programmed to predict the response to different courses of treatment, including immunotherapy, which will classify the corresponding patient subset group to which the treatment would be most beneficial. AI streamlined processes to accurately and systematically determine successful treatments, an innovation that will enable personalized cancer care in one embodiment.

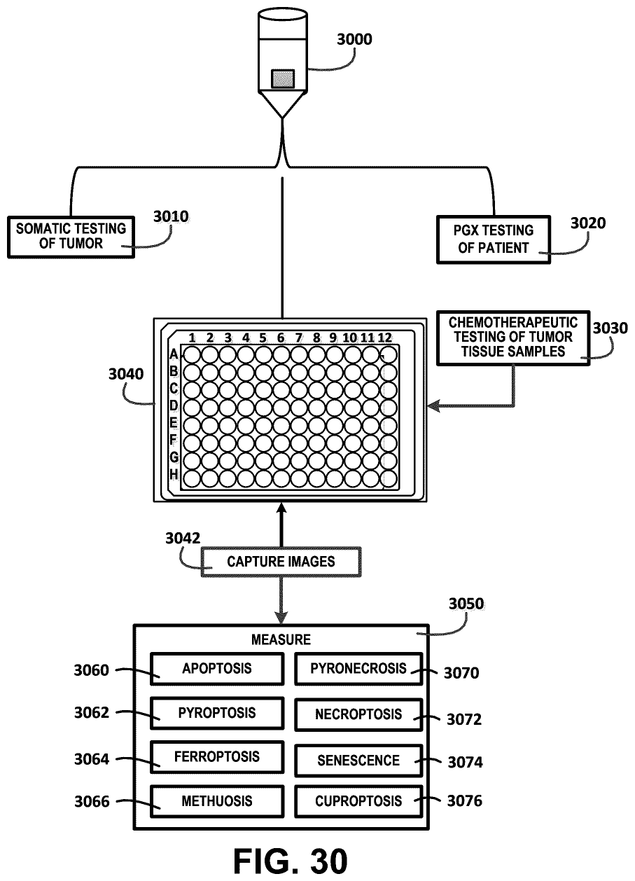

FIG. 30 shows a block diagram of an overview flow chart of programmed cell death overcoming apoptosis resistance of one embodiment. FIG. 30 shows a treatment drug 3000 used for somatic testing of tumor 3010 and PGx testing of patient 3020. The chemotherapeutic testing of tumor tissue samples 3030 is performed in a 96-cell microplate 3040, and a camera is used to capture images 3042 of the 96-cell microplate 3040. The testing and captured images are used to measure 3050 various forms of cancer cell death, including apoptosis 3060, pyroptosis 3062, ferroptosis 3064, methuosis 3066, pyronecrosis 3070, necroptosis 3072, senescence 3074, and other cell death, including the newly described cuproptosis 3076 in the forms of one embodiment.

Programmed cell death, overcoming apoptosis resistance, necroptosis serves as an alternative mode of programmed cell death. Pyronecrosis is a proinflammatory cell death. Ferroptosis is an intracellular iron-dependent form of cell death that is distinct from apoptosis, necrosis, and autophagy. Cuproptosis 3076 is a copper-driven form of cell death that exerts an inhibiting role in tumor growth and may open the door for the treatment of chemotherapy-insensitive tumors. Methuosis is a unique form of non-apoptotic cell death triggered by alterations in the trafficking of clathrin-independent endosomes. Pyroptosis is a type of programmed cell death characterized by the activation of inflammatory caspases. Senescent cells are unique in that they eventually stop multiplying but do not die off when they should have in one embodiment.

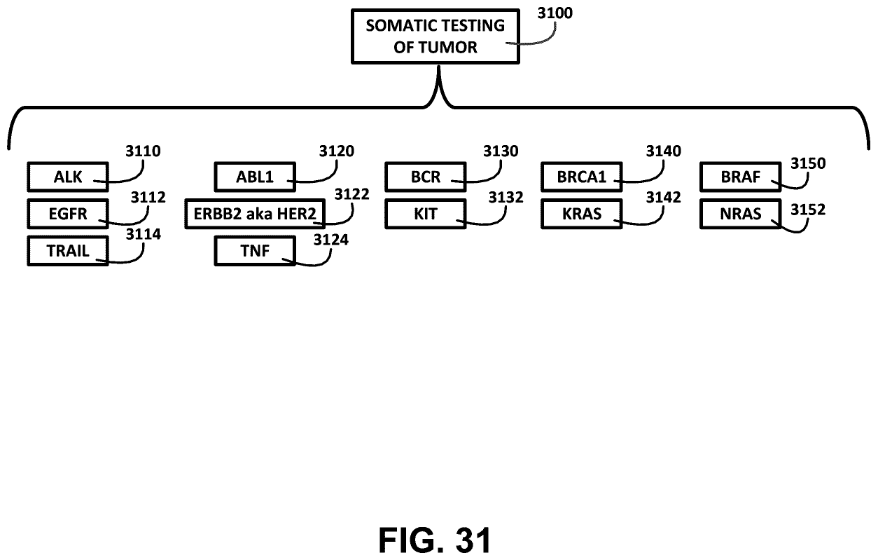

FIG. 31 shows a block diagram of an overview flow chart of somatic gene variants of one embodiment. FIG. 31 shows that necroptosis serves as an alternative mode of programmed cell death, overcoming apoptosis resistance. Pyronecrosis is a proinflammatory cell death. Methuosis is a unique form of non-apoptotic cell death triggered by alterations in the trafficking of clathrin-independent endosomes. Pyroptosis is a type of programmed cell death characterized by the activation of inflammatory caspases. Ferroptosis is an intracellular iron-dependent form of cell death that is distinct from apoptosis, necrosis, and autophagy. In contrast to ferroptosis, which is iron-dependent, cuproptosis 3076 of FIG. 30 is a copper-driven death pathway; cuproptosis 3076 of FIG. 30 needs lipoylated-enzyme aggregation and is iron-sulfur depletion-driven, not lipid-peroxide-driven; iron chelators don't rescue, but lipoate synthesis inhibitors do rescue. Senescent cells are unique in that they eventually stop multiplying but do not die off when they should have in one embodiment.

FIG. 31 shows somatic testing of tumor 3100 for the presence of and possible treatment of ALK 3110, EGFR 3112, TRAIL 3114, ABL1 3120, ERBB2 aka HER2 3122, TNF 3124, BCR 3130, KIT 3132, BRCA1 3140, KRAS 3142, BRAF 3150, and NRAS 3122. Somatic gene variants are the most common cause of cancer, occurring from damage to genes in an individual cell during a person's life, of one embodiment.

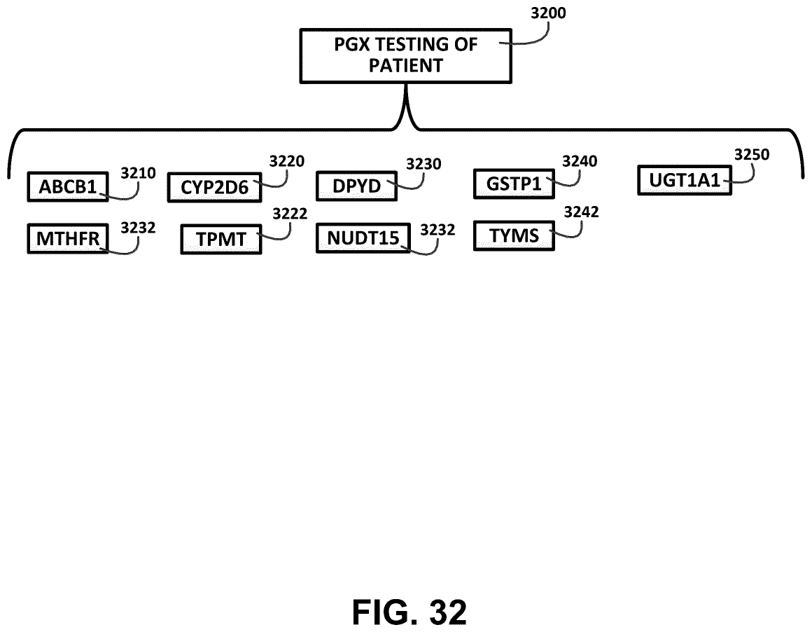

FIG. 32 shows for illustrative purposes only an example of personalized chemotherapy drug dosing of one embodiment. FIG. 32 shows PGx testing of a patient 3200. The PGx testing detects certain genes that can affect the patient's response to a medication and may be affected by the dosing of the medication. PGx testing detects at least the following genes: ABCB1 3210, MTHFR 3114, CYP2D6 3220, TPMT 3222, DPYD 3230, NUDT15 3232, GSTP1 3240, TYMS 3242, and UGT1A1 3250.

Many cancer patients who receive chemotherapy experience adverse drug effects. PGx (Pharmacogenomics) is the study of how human genes can impact the way a body reacts to certain prescription or over-the-counter drugs. Genes, which are inherited from parents, hold information that determines things like eye color and blood type. These genes can also affect how a person processes and responds to medications. Based on a unique genetic makeup, some drugs might work faster or slower, and a person might experience more or fewer side effects. Pharmacogenomics (PGx) has promise to personalize chemotherapy drug dosing to maximize efficacy and safety of one embodiment.

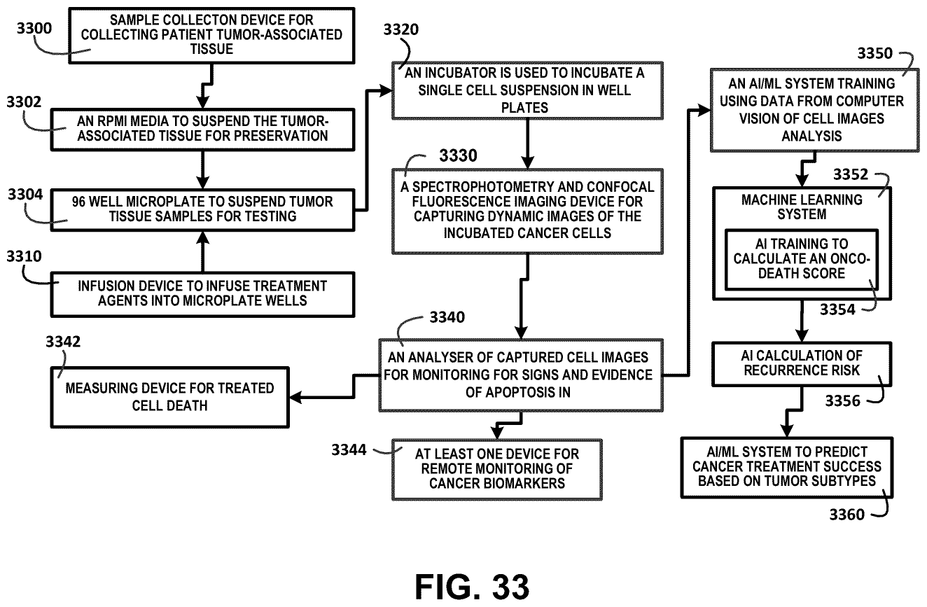

FIG. 33 shows for illustrative purposes only an example of personalized chemotherapy drug dosing of one embodiment. FIG. 33 shows a sample collection device for collecting patient tumor-associated tissue 3300. The collection device may be used in an operating room to collect live tumor cells. An RPMI media to suspend the tumor-associated tissue for preservation 3302, is used to keep the tumor tissue alive during shipping to a laboratory. A 96-well microplate to suspend tumor tissue samples for testing, 3304, is used to segregate cells for exposure to different treatment drugs alone or in combination. An infusion device to infuse treatment agents into microplate wells 3310 is used to infuse a predetermined amount of each treatment agent into the microplate wells. After infusion, an incubator is used to incubate a single cell suspension in well plates 3320 to promote cell growth and measure the effectiveness of each treatment drug in causing the death of the tumor cancer cells. A spectrophotometry and confocal fluorescence imaging device for capturing dynamic images of the incubated cancer cells 3330. An analyzer of captured cell images for monitoring for signs and evidence of apoptosis in 3340 of the tumor cells. A measuring device for treated cell death 3342 is used to measure the sequential pictures taken every 5 minutes over 48 hours. At least one device for remote monitoring of cancer biomarkers 3344 detects and identifies biomarkers that indicate the level of apoptosis of the cancer cells.

An AI/ML system training using data from computer vision of cell images analysis 3350 builds a collection of data that provides a historical record of the cell death for each treatment agent. The machine learning 124 system 3352 can be used to forecast the effectiveness of different treatments for different subtypes of cancer cells. AI training to calculate an oncological-death score 3354 uses computer vision, numerical, or symbolic information of the captured images that is interpreted as an oncological-death score. The accumulation of the image analysis and computer vision interpretations allows an AI calculation of recurrence risk 3356 for a particular cancer, for a specific patient. The oncological-death score and AI/ML system learning allow the AI/ML system to predict cancer treatment success based on tumor subtypes 3360.

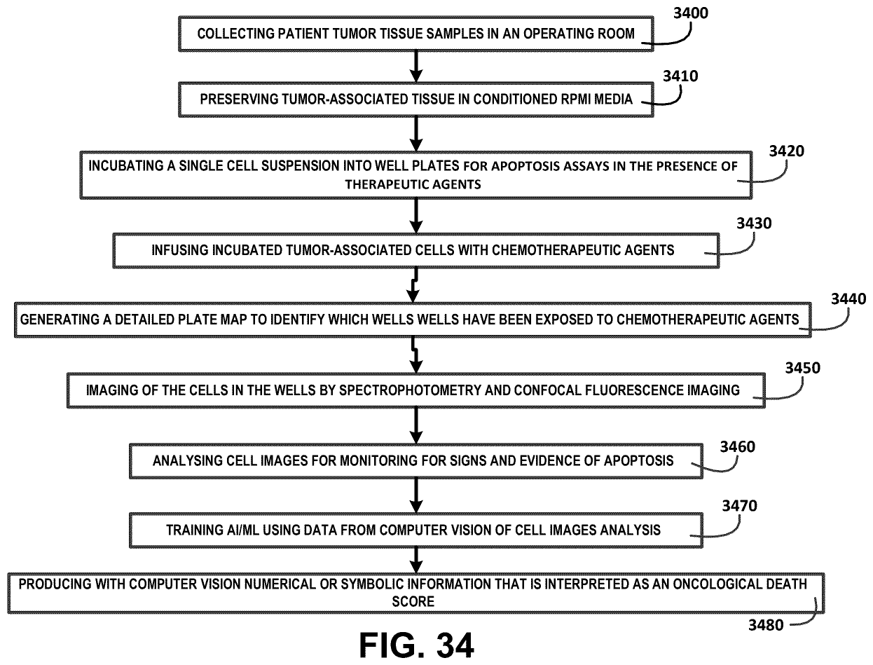

FIG. 34 shows a block diagram of an overview of the APOP assay for active tumor cell death in one embodiment. FIG. 34 shows an example of collecting patient tumor tissue samples in an operating room 3400. The collection process includes preserving tumor-associated tissue in conditioned RPMI media 3410, incubating a single cell suspension in well plates for apoptosis assays in the presence of therapeutic agents 3420, and infusing incubated tumor-associated cells with chemotherapeutic agents 3430 and generating a detailed plate map to identify which wells have been exposed to chemotherapeutic agents 3440. Imaging of the cells in the wells by spectrophotometry and confocal fluorescence imaging 3450. Analyzing cell images for monitoring for signs and evidence of apoptosis 3460. Training AI/ML using data from computer vision of cell images analysis 3470 and producing with computer vision numerical or symbolic information that is interpreted as an oncological death score 3480.

A plurality of treatment drugs is used in somatic testing of a tumor. Tumor cells are placed in a 96-cell microplate. The treatment drug is added to the cells of the microplate, and photographic images are captured periodically to measure the effectiveness of the treatment drug in causing the tumor cancer cell death, including various forms, including apoptosis, pyroptosis, ferroptosis, cuproptosis, methuosis, pyronecrosis, necroptosis, and senescence. The somatic testing of tumor cells results is accumulated in an assay for active tumor cell death. The somatic testing of tumor cells results include measurement of plasma membrane blebbing, measurement of cytochrome C, measurement of annexin V, and measurement of caspase(s). The assay for active tumor cell death results is processed using machine learning 124. The machine learning 124 analyzed and compared assay data, which is used for training of AI to calculate an oncological-death score based on multiple parameters that are measured with digital imaging of monolayers. The monitoring of cancer biomarkers in saliva and serum using a multiplex microarray is also added to the assay data to further define the multiple parameters that are measured. AI calculation of recurrence risk based on multiple secreted parameters increases the accuracy of the predictions of outcomes based on treatments and types of cancer of one embodiment.

The process steps include a procedure for collecting samples using a collection device. A disruption device and protocol for somatic testing to detect EGFR, KRAS, BRAF, TP53, PIK3, PTEN, others, and PGx testing. The steps continue with a treatment matrix and incubation of the test microbiology sample 102 of FIG. 1 and infusion of various treatment drugs alone and in combination. The microplate testing includes the collection of images and fluorescent measurements. The process further includes an analysis module of images and intensities. The analysis is used to formulate a set of computer-readable instructions of AI/ML to generate an oncological death score for each treatment and for each type of cancer cells. The processes include at least one device for remote monitoring of soluble cancer markers, for example, cancer biomarkers of one embodiment.

A collected sample with a collection device is used, where each sample is collected in the operating room as a biopsy is taken for traditional histology and grading. The tumor-associated tissue is preserved and shipped live to the laboratory in conditioned RPMI media of one embodiment. A disruption device and protocol in the laboratory; once the tissue arrived are then disrupted into a single cell suspension for tissue culture into 96-well plates at 37 degrees C. and 5% CO₂.

The 96-well plates with suspended cells are incubated until the cells have settled and establish a monolayer of tumor-associated cells that will be used for apoptosis assays in the presence of therapeutic agents of one embodiment. A chemotherapeutic treatment matrix and incubation device uses 96-well monolayers of tumor-associated cells, which are incubated at 37 degrees C. and 5% CO₂ with chemotherapeutic agents at pre-determined concentrations and combinations according to NCCN guidelines. The NCCN guidelines are published by the National Comprehensive Cancer Network® (NCCN®), which is a not-for-profit alliance of 33 leading cancer centers devoted to patient care, research, and education. A detailed plate map is generated of which wells have been exposed to chemotherapeutic agents and at which concentration or combination of concentrations in one embodiment.

A collection of images and fluorescent material is obtained. The collection of images and fluorescent measurements is taken at 5-minute intervals, dynamic imaging of the cell monolayers by spectrophotometry, and confocal fluorescence imaging for evidence of cell blebbing, DNA fragmentation, expression of caspase, annexin V, and cytochrome C. Cells are dynamically imaged every 5 minutes over 48 hours. There are 576 sequential images that are captured of each well, yielding 55,296 images per 96-well plate. The images are fed into analysis modules to establish intensity curves of fluorescence and quantify cell blebbing and DNA fragmentation of one embodiment.

An analysis module of images and intensities. The analysis module of images and intensities includes 55,296 images that are captured per plate. Automated image analysis is needed due to the image volume and multiparametric data. The process uses aspects of fluorescent quantitation, and the images are gated and analyzed for signs and evidence of apoptosis. The process includes multiparametric training of AI/ML, which is performed using data from DNA fragmentation, cell blebbing, and fluorescent signal using aspects of computer vision. The computer vision utilizes methods for acquiring, processing, analyzing, and understanding digital images, and the extraction of high-dimensional data from the real world to produce numerical or symbolic information that is interpreted as an oncological-death score of one embodiment.

A set of computer-readable instructions of AI/ML generates an oncological death score. The set of computer-readable instructions of AI/ML to generate the oncological-death score uses multimodal machine learning 124 and deep learning for image analysis training. Data generated from multiparametric analysis of cell death from the 55,296 images is fed into an AI/ML module to calculate a metric called the oncological-death, which is a measurement of the amount of cell death that is generated by the chemotherapeutic exposure(s). The oncological-death score report is provided to oncologists and surgeons that defines and details the chemotherapeutic or combination of chemotherapeutics that are most efficient at generating cell death in patient-derived cells of one embodiment.

A device for remote monitoring of soluble cancer markers. The device for remote monitoring of soluble cancer markers is configured for post-treatment monitoring of cancer biomarkers performed using piezoelectric biosensors. Post-treatment monitoring of cancer biomarkers is performed by proteomic analysis, measuring proteins including Ki-67, sPD-L1, VEGF, MMP-9, CXCR4 receptor, CEA, AFP, β-HCG, CA15-3, CA19-9, CA27.29, and CA125, among others, that are specific for cancer progression. Monitoring of these markers allows for early detection of possible recurrence and/or treatment failure of one embodiment.

In one aspect, the invention includes a method for quantitatively tracking neoplastic progression in a subject, the method comprising: (a) obtaining at sequential time points a peripheral-blood biospecimen of 2-20 mL, optionally stabilized with an anticoagulant and a cell-stabilizing preservative; (b) within 6 hours of phlebotomy, separating plasma by double-spin centrifugation to yield a cell-free fraction substantially depleted of leukocytes; (c) extracting total cell-free nucleic acids with a silica-membrane or magnetic-bead protocol optimized for fragments between 90 bp and 220 bp; (d) enriching circulating tumor DNA (ctDNA) by target-specific hybrid-capture, multiplex PCR, or size-selection; (e) digitally quantifying tumor-specific sequence variants—comprising at least one somatic single-nucleotide variant, insertion/deletion, structural rearrangement, methylation signature, or fragmentomic pattern, using next-generation sequencing at≥10 000× unique molecular coverage or droplet digital PCR with a limit of detection ≤0.01% variant allele frequency; (f) computing a progression index by comparing the absolute or relative ctDNA burden, molecular residual-disease status, or variant-derived fragmentomics across two or more time points; and (g) reporting an increase in said index that exceeds a predetermined threshold (e.g., a ≥1.5-fold rise in variant allele fraction or re-emergence of any previously cleared mutation) as predictive of radiologic or clinical progression months prior to conventional imaging. Optionally, the method integrates complementary metrics, including circulating tumor-cell enumeration or protein biomarker levels, to refine sensitivity and specificity. The described workflow enables real-time therapeutic adaptation, early relapse detection, and improved patient stratification in oncology care.

Disclosed herein are compositions and methods for quantitatively monitoring solid-tumor progression using a multimoal biomarker panel that integrates (i) proliferation index Ki-67; (ii) angiogenic factor VEGF-A and optional soluble PD-L1; (iii) invasion-metastasis mediators MMP-9 and CXCR4; (iv) liquid-biopsy dynamics comprising circulating-tumour-DNA (ctDNA) allele fraction and circulating-tumour-cell (CTC) count or cluster phenotype; (v) metabolic-stress signature (13 cuproptosis-related genes including FDX1 and LIAS); and (vi) progression-associated non-coding RNAs, exemplified by IncRNA PVT1 and exosomal RNAs TBILA, H19 and MALAT1. In one embodiment, values for at least three markers from different biological classes are obtained from a patient specimen (tissue, blood, or exosomes), normalized, and combined into a weighted progression risk score that predicts stage up-grading, metastatic spread, or relapse. The score results guide therapeutic decisions—including timing of adjuvant chemotherapy, anti-angiogenic, or immune-checkpoint therapy—and enable real-time adaptation of treatment regimens to improve progression-free and overall survival.

In one aspect, the invention includes and provides a method for enhancing cancer therapy selection and optimizing patient outcomes through the genotypic analysis of the haptoglobin (Hp) gene. The method comprises identifying a subject's Hp genotype (Hp1-1, Hp2-1, or Hp2-2) using a molecular assay and unique primers and probes that allow for the detection of the exon duplication that is a hallmark of Hp2-2, wherein the presence of the Hp2-2 genotype is predictive of elevated systemic oxidative stress, chronic inflammation, and immune dysregulation. These pathophysiologic features contribute to fibrotic stromal remodeling and immune suppression within the tumor microenvironment, thereby reducing the efficacy of cancer therapies, including immune checkpoint inhibitors, adoptive cell transfer, and chemotherapy. By stratifying patients according to their Hp genotype, clinicians can adjust therapeutic regimens, implement adjunctive anti-inflammatory or antifibrotic interventions, or prioritize alternative treatment modalities that are less sensitive to immune evasion. The method also provides a diagnostic rationale for incorporating haptoglobin genotyping into cancer precision medicine workflows, particularly for patients exhibiting comorbid chronic inflammatory states, including periodontal disease, metabolic syndrome, or autoimmune conditions.

The present invention includes methods and compositions for enhancing the efficacy of anti-cancer therapies in collagen-rich desmoplastic tumors. In one aspect, the invention provides a combination therapy comprising (a) a conventional anti-tumor agent selected from chemotherapeutics, targeted small molecules, or immune checkpoint inhibitors, and (b) an extracellular matrix, modulating agent capable of reducing collagen crosslinking, degrading collagen fibers, or inhibiting collagen-mediated signaling. Combination therapy is particularly suited for the treatment of tumors characterized by high collagen content and stiffness, including but not limited to pancreatic ductal adenocarcinoma, breast carcinoma, and pulmonary adenocarcinoma. In a preferred embodiment, the ECM-modulating agent is a lysyl oxidase (LOX/LOXL) inhibitor, collagenase, an anti-TGF-β biologic, or an integrin/DDR antagonist.

Administration of the combination therapy results in (i) decreased extracellular matrix stiffness, (ii) improved penetration and distribution of the anti-tumor agent within the tumor mass, and (iii) enhanced tumor cell apoptosis and immune cell infiltration, thereby overcoming collagen-mediated treatment resistance.

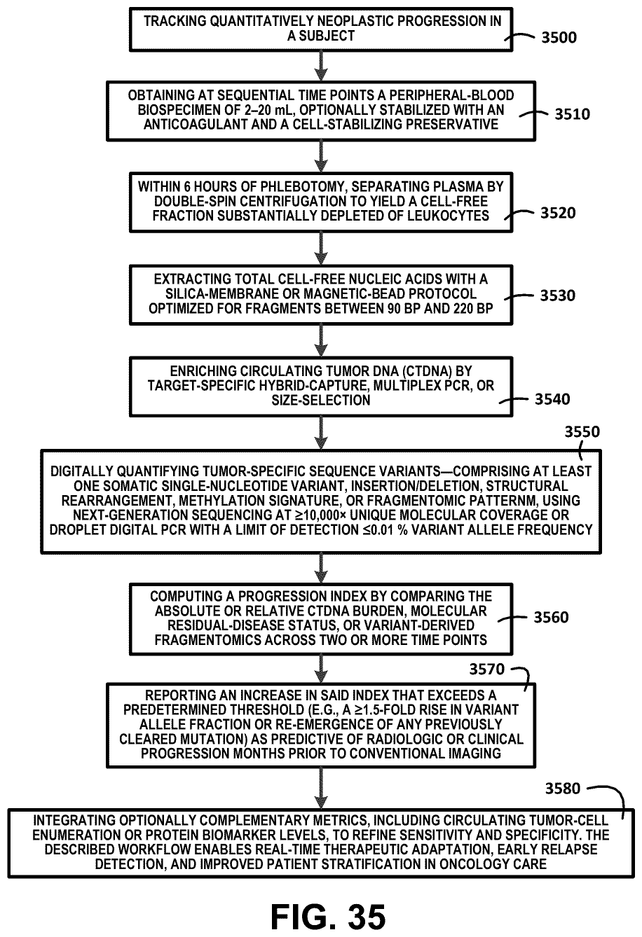

FIG. 35 shows a block diagram of an overview flow chart of a method for quantitatively tracking neoplastic progression in a subject of one embodiment. FIG. 35 shows tracking quantitatively neoplastic progression in a subject 3500 obtaining at sequential time points a peripheral-blood biospecimen of 2-20 ml, optionally stabilized with an anticoagulant and a cell-stabilizing preservative 3510 within 6 hours of phlebotomy, separating plasma by double-spin centrifugation to yield a cell-free fraction substantially depleted of leukocytes 3520. Extracting total cell-free nucleic acids with a silica-membrane or magnetic-bead protocol optimized for fragments between 90 bp and 220 bp 3530. Enriching circulating tumor DNA (ctdna) by target-specific hybrid-capture, multiplex PCR, or size-selection 3540.

Quantifying digitally tumor-specific sequence variants, comprising at least one somatic single-nucleotide variant, insertion/deletion, structural rearrangement, methylation signature, or fragmentomic pattern, using next-generation sequencing at≥10,000× unique molecular coverage or droplet digital PCR with a limit of detection ≤0.01% variant allele frequency 3550. Computing a progression index by comparing the absolute or relative ctdna burden, molecular residual-disease status, or variant-derived fragmentomics across two or more time points 3560; and reporting an increase in said index that exceeds a predetermined threshold (e.g., a≥1.5-fold rise in variant allele fraction or re-emergence of any previously cleared mutation) as predictive of radiologic or clinical progression months prior to conventional imaging 3570. Integrating optionally complementary metrics, including circulating tumor-cell enumeration or protein biomarker levels, to refine sensitivity and specificity. The described workflow enables real-time therapeutic adaptation, early relapse detection, and improved patient stratification in oncology care 3580.

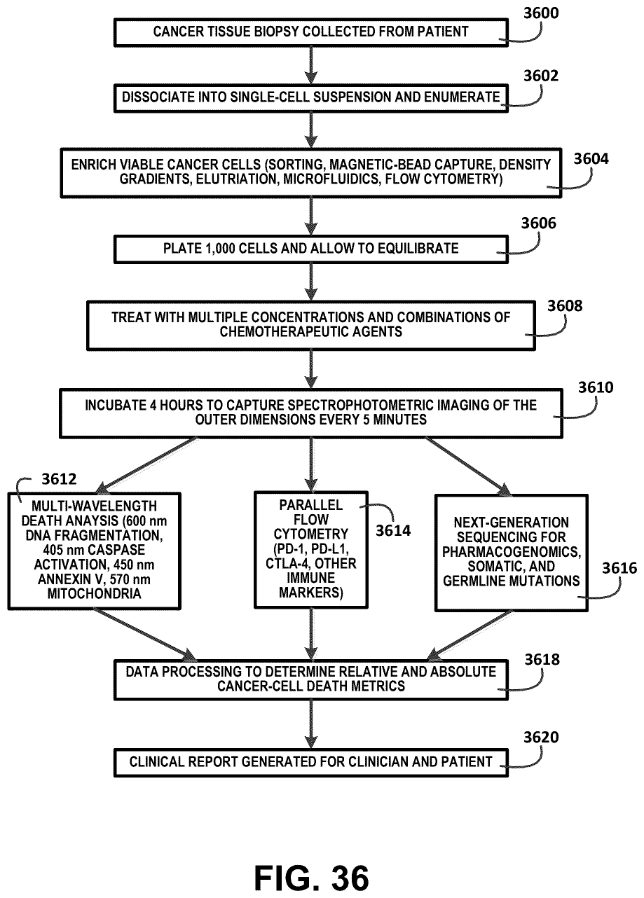

FIG. 36 shows a block diagram of an overview of relative and absolute cancer-cell death metrics of one embodiment. FIG. 36 shows a cancer tissue biopsy collected from patient 3600. Dissociate into a single-cell suspension and enumerate 3602. Enrich viable cancer cells (sorting, magnetic-bead capture, density gradients, elutriation, microfluidics, flow cytometry) 3604. Plate 1,000 cells and allow to equilibrate 3606. Then treat with multiple concentrations and combinations of chemotherapeutic agents 3608. Incubate for 4 hours to capture spectrophotometric imaging of the outer dimensions every 5 minutes 3610. Multi-wavelength death analysis (600 nm DNA fragmentation, 405 nm caspase activation, 450 nm annexin v, 570 nm mitochondria 3612. Parallel flow cytometry (PD-1, PD-L1, CTLA-4, other immune markers) 3614. Next-generation sequencing for pharmacogenomics, somatic, and germline mutations 3616. Data processing to determine relative and absolute cancer-cell death metrics 3618. Clinical report generated for clinician and patient 3620 of one embodiment.

The sensor array is configured to monitor environmental conditions affecting the microbiology sample 102 of FIG. 1 live cancer cells contained in the microplate. The array includes multiple sensors, including temperature sensors, humidity sensors, and chemical detectors, each generating data in real time. These sensors capture variations in the local environment that could influence cell growth or measurement accuracy. The sensor array is coupled to the control logic, enabling the system to adjust conditions or measurement parameters based on sensor input. By integrating multiple sensing elements into a unified system, the sensor array provides a comprehensive data set that enhances the reliability of the optical density measurements.

The control logic coordinates the operation of the optical density measurement device 111, the optical sensor, the illumination source, and the sensor array. It processes incoming signals, manages timing of measurement cycles, and ensures synchronization between data capture and storage. The control logic is configured to execute programmed instructions, which include initiating image capture at predetermined intervals, activating the illumination source, and adjusting measurement parameters based on input from the sensor array. It also directs communication between the processing unit and peripheral components, ensuring accurate data flow throughout the system.

The data analysis module is configured to interpret the measurements generated by the optical sensor and the processing unit. It applies computational methods to identify trends, quantify cell population changes, and detect growth phases of the microbiology live cancer cells. The module may include algorithms for curve fitting, statistical averaging, and predictive modeling, which transform raw measurements into actionable results. Data from the sensor array is incorporated into these analyses to account for environmental influences, improving the accuracy of the population measurements. The data analysis module outputs processed results to the storage medium and to the wireless communication interface for further use.

The wireless communication interface enables bidirectional data transfer between the optical density measurement device 111 and external devices including a mobile phone, computer, or remote database. It is configured to transmit raw and processed measurement data, environmental sensor readings, and analysis results generated by the processing unit. The wireless communication interface also receives control inputs, including updated measurement protocols or software instructions, which are executed by the control logic. This communication capability allows the measurement system to operate as part of a distributed network, facilitating real-time monitoring and remote management of experiments.

The foregoing has described the principles, embodiments, and modes of operation of the present invention. However, the invention should not be construed as being limited to the embodiments discussed. The above-described embodiments should be regarded as illustrative rather than restrictive, and it should be appreciated that variations may be made in those embodiments by workers skilled in the art without departing from the scope of the present invention as defined by the following claims.

What is claimed is:

1. An optical density measurement and testing device, comprising
a purification device configured to receive a microbiology sample and distinguish and separate live cancer cells from dead cancer cells and non-cancer cells in the microbiology sample;
at least one microplate configured to receive the separated live cancer cells from the purification device;
a drug addition device coupled to the at least one microplate and configured to infuse at least one drug treatment into the live cancer cells;
a drug dosage sequencer device coupled to the drug addition device and configured to control administration amounts of different treatment drug dosages of the infused at least one drug treatment into the live cancer cells;
an optical density measurement device having an optical sensor configured to capture and record high-resolution images of the separated live cancer cells from the microplate at predetermined different intervals to measure a population of the live cancer cells at the predetermined different intervals;
an optical spectrophotometric reader coupled to the optical density measurement device and configured to measure the population of the living cells after the at least one drug treatment is infused into the live cancer cells;
a flow cytometry device coupled to the optical spectrophotometric reader, the flow cytometry device configured to measure fluorescent intensities of immune checkpoint markers and cancer tumor antigens, including PD-1, PD-L1, and CTLA-4, expressed in the live cancer cells, and to use the measured intensities to quantify levels of immune antigen at different predetermined intervals, thereby generating immune system activation profiles induced by the at least one drug treatment;
a first processor of a computer coupled to the optical spectrophotometric reader, the first processor configured to analyze the population measurements of the live cancer cells captured at different predetermined intervals and to determine rates of cell death of the live cancer cells over a predetermined period of time based on changes in population growth of the live cancer cells;
a second processor of the computer coupled to the first processor, the second processor configured to analyze the fluorescent intensities of the immune checkpoint markers and the cancer tumor antigens together with the rates of cell death of the live cancer cells to assess immune antigen stimulation and release of immune antigens to an immune system of a living organism, thereby generating integrated profiles of drug response and activation of the immune system; and
a computer application operating on the computer and coupled to the first and second processors, the computer application configured to compare the determined rates of cell death of the live cancer cells, the release of immune antigens, the integrated profiles of drug response and the activation of the immune system to known patient-specific information including known genetic markers, known drug resistances, and known allergies associated with the at least one drug treatment to generate interactive treatment options for clinician review that balances therapeutic effectiveness with patient tolerability to create personalized drug treatment recommendations for the living organism.

2. The optical density measurement and testing device of claim 1, wherein the purification device is further configured to disrupt the microbiology sample for somatic testing and for culture apoptosis assays in a presence of therapeutic agents.

3. The optical density measurement and testing device of claim 1, further comprising a sensor array coupled to the microplate configured to monitor environmental conditions affecting the microbiology sample live cancer cells contained in the microplate.

4. The optical density measurement and testing device of claim 1, wherein the administration of different dosages of the treatment drugs includes administration of sub-therapeutic and supra-therapeutic concentrations to permit comparative evaluation of drug activity across a range of conditions.

5. The optical density measurement and testing device of claim 1, further comprising at least one analytical device coupled to the computer configured to detect, identify, and monitor soluble cancer markers.

6. The optical density measurement and testing device of claim 1, wherein the second processor is further configured to calculate an oncological-death score with chemotherapeutic and combination of chemotherapeutics most efficient at generating cell death in patient-derived cells.

7. The optical density measurement and testing device of claim 1, wherein the optical spectrophotometric reader is further configured to capture images at predetermined different intervals to calculate concentrations of both living cells and dead cells of the microbiology sample to indicate efficacy of a treatment plan over predetermined time intervals.

8. An optical density measurement and testing device, comprising:
a purification device configured to receive a microbiology sample and distinguish and separate live cancer cells from dead cancer cells and non-cancer cells in the microbiology sample;
at least one microplate configured to receive the separated live cancer cells from the purification device;
a drug addition device coupled to the at least one microplate and configured to infuse at least one drug treatment into the live cancer cells;
a drug dosage sequencer device coupled to the drug addition device and configured to control administration amounts of different treatment drug dosages of the infused at least one drug treatment into the live cancer cells;
an optical density measurement device having an optical sensor configured to capture and record high-resolution images of the separated live cancer cells from the microplate at predetermined different intervals to measure a population of the live cancer cells at the predetermined different intervals;
an optical spectrophotometric reader coupled to the optical density measurement device and configured to measure the population of the living cells after the at least one drug treatment is infused into the live cancer cells;
a flow cytometry device coupled to the optical spectrophotometric reader, the flow cytometry device configured to measure fluorescent intensities of immune checkpoint markers and cancer tumor antigens, including PD-1, PD-L1, and CTLA-4, expressed in the live cancer cells, and to use the measured intensities to quantify levels of immune antigen at different predetermined intervals, thereby generating immune system activation profiles induced by the at least one drug treatment;
a first processor of a computer coupled to the optical spectrophotometric reader, the first processor configured to analyze the population measurements of the live cancer cells captured at different predetermined intervals and to determine rates of cell death of the live cancer cells over a predetermined period of time based on changes in population growth of the live cancer cells;
a second processor of the computer coupled to the first processor, the second processor configured to analyze the fluorescent intensities of the immune checkpoint markers and the cancer tumor antigens together with the rates of cell death of the live cancer cells to assess immune antigen stimulation and release of immune antigens to an immune system of a living organism, thereby generating integrated profiles of drug response and activation of the immune system;
a computer application operating on the computer and coupled to the first and second processors, the computer application configured to compare the determined rates of cell death of the live cancer cells, the release of immune antigens, the integrated profiles of drug response and the activation of the immune system to known patient-specific information including known genetic markers, known drug resistances, and known allergies associated with the at least one drug treatment to generate interactive treatment options for clinician review that balances therapeutic effectiveness with patient tolerability to create personalized drug treatment recommendations for the living organism; and
an artificial intelligence processor coupled to the computer and configured to calculate cell viability curves, apoptosis kinetics, genetic marker associations, and drug dosage response profiles for training large-scale datasets to recognize apoptosis patterns and predict treatment outcomes.

9. The optical density measurement and testing device of claim 8, wherein the purification device is further configured to disrupt the microbiology sample for somatic testing and for culture apoptosis assays in a presence of therapeutic agents.

10. The optical density measurement and testing device of claim 8, further comprising a post-treatment monitoring device to monitor cancer biomarkers by proteomic analysis that includes measuring proteins including Ki-67, sPD-L1, VEGF, MMP-9, CXCR4 receptor, CEA, AFP, β-HCG, CA15-3, CA19-9, CA27.29, and CA125, that are specific for cancer progression.

11. The optical density measurement and testing device of claim 8, wherein the administration of different dosages of the treatment drugs includes administration of sub-therapeutic and supra-therapeutic concentrations to permit comparative evaluation of drug activity across a range of conditions.

12. The optical density measurement and testing device of claim 8, wherein the artificial intelligence processor includes at least one analysis module to evaluate multiparametric datasets from optical density, fluorescent imaging, and biomarker measurements.

13. The optical density measurement and testing device of claim 8, wherein the second processor is further configured to calculate an oncological-death score with chemotherapeutic and combination of chemotherapeutics most efficient at generating cell death in patient-derived cells.

14. The optical density measurement and testing device of claim 8, wherein the optical spectrophotometric reader is further configured to capture images at predetermined different intervals to calculate concentrations of both living cells and dead cells of the microbiology sample to indicate efficacy of a treatment plan over predetermined time intervals.

15. An optical density measurement and testing device, comprising:
a purification device configured to receive a microbiology sample and distinguish and separate live cancer cells from dead cancer cells and non-cancer cells in the microbiology sample;
at least one microplate configured to receive the separated live cancer cells from the purification device;
a drug addition device coupled to the at least one microplate and configured to infuse at least one drug treatment into the live cancer cells;
a drug dosage sequencer device coupled to the drug addition device and configured to control administration amounts of different treatment drug dosages of the infused at least one drug treatment into the live cancer cells;
an optical density measurement device having an optical sensor configured to capture and record high-resolution images of the separated live cancer cells from the microplate at predetermined different intervals to measure a population of the live cancer cells at the predetermined different intervals;
an optical spectrophotometric reader coupled to the optical density measurement device and configured to measure the population of the living cells after the at least one drug treatment is infused into the live cancer cells;
a flow cytometry device coupled to the optical spectrophotometric reader, the flow cytometry device configured to measure fluorescent intensities of immune checkpoint markers and cancer tumor antigens, including PD-1, PD-L1, and CTLA-4, expressed in the live cancer cells, and to use the measured intensities to quantify levels of immune antigen at different predetermined intervals, thereby generating immune system activation profiles induced by the at least one drug treatment;
a first processor of a computer coupled to the optical spectrophotometric reader, the first processor configured to analyze the population measurements of the live cancer cells captured at different predetermined intervals and to determine rates of cell death of the live cancer cells over a predetermined period of time based on changes in population growth of the live cancer cells;
a second processor of the computer coupled to the first processor, the second processor configured to analyze the fluorescent intensities of the immune checkpoint markers and the cancer tumor antigens together with the rates of cell death of the live cancer cells to assess immune antigen stimulation and release of immune antigens to an immune system of a living organism, thereby generating integrated profiles of drug response and activation of the immune system;
a computer application operating on the computer and coupled to the first and second processors, the computer application configured to compare the determined rates of cell death of the live cancer cells, the release of immune antigens, the integrated profiles of drug response and the activation of the immune system to known patient-specific information including known genetic markers, known drug resistances, and known allergies associated with the at least one drug treatment to generate interactive treatment options for clinician review that balances therapeutic effectiveness with patient tolerability to create personalized drug treatment recommendations for the living organism, wherein the computer application includes a graphical user interface configured to display the interactive treatment options; and
an artificial intelligence processor coupled to the computer and configured to calculate cell viability curves, apoptosis kinetics, genetic marker associations, and drug dosage response profiles for training large-scale datasets to recognize apoptosis patterns and predict treatment outcomes.

16. The optical density measurement and testing device of claim 15, wherein the purification device is further configured to disrupt the microbiology sample for somatic testing and for culture apoptosis assays in a presence of therapeutic agents.

17. The optical density measurement and testing device of claim 15, further comprising at least one analytical device coupled to the computer configured to detect, identify, and monitor soluble cancer markers.

18. The optical density measurement and testing device of claim 15, wherein the optical spectrophotometric reader is further configured to capture images at predetermined different intervals to calculate concentrations of both living cells and dead cells of the microbiology sample to indicate efficacy of a treatment plan over predetermined time intervals.

19. The optical density measurement and testing device of claim 15, wherein the second processor is further configured to calculate an oncological-death score with chemotherapeutic and combination of chemotherapeutics most efficient at generating cell death in patient-derived cells.

20. The optical density measurement and testing device of claim 15, further comprising a sensor array coupled to the microplate configured to monitor environmental conditions affecting the microbiology sample live cancer cells contained in the microplate.
